(19) United States
(12) Patent Application Publication
(10) Pub. No.: US 2011/0046724 A1
(43) Pub. Date: Feb. 24, 2011

(54) Expansible Biocompatible Coats Comprising a Biologically Active Substance

(75) Inventors: Torsten Heilmann, Bad Klosterlausnitz (DE); Christian Richter, Oberwurschnitz (DE); Sabine Post, Berlin (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Avidal Vascular GMBH, Halle (Saale) (DE)

(21) Appl. No.: 12/934,762

(22) PCT Filed: Mar. 31, 2009
(86) PCT No.: PCT/EP2009/002348

§ 371 Date: Nov. 5, 2010

Related U.S. Application Data
(60) Provisional application No. 61/040,967, filed on Mar. 31, 2008.

(30) Foreign Application Priority Data
Mar. 31, 2008 (EP) 08006483.5

(51) Int. Cl. A61F 2/82; A61M 29/00; B05D 3/12

(52) U.S. Cl. 623/1.46; 604/96.01; 427/2.1

(57) ABSTRACT

The present invention relates to an expansible hollow part, having at least one opening, which consists of an elastic biocompatible material and which comprises at least one biologically active substance and, optionally at least one matrix compound. The invention also provides a method of producing said expansible hollow part, a medical device covered at least partially with said hollow part, a kit-of-parts comprising said hollow part of the invention and the use of said hollow part as a therapeutic device and for protecting a medical device.

The present invention relates to an expansible hollow part, having at least one opening, which consists of an elastic biocompatible material and which comprises at least one biologically active substance and, optionally at least one matrix compound. The invention also provides a method of producing said expansible hollow part, a medical device covered at least partially with said hollow part, a kit-of-parts comprising said hollow part of the invention and the use of said hollow part as a therapeutic device and for protecting a medical device.

### BACKGROUND OF THE INVENTION

At present, diseased vessels, vascular constrictions and certain organ failures, e.g. due to a stricture can be treated by systemic administration of pharmaceuticals and by expanding and supporting an affected vessel segment or organ lumen by introducing and expanding a balloon catheter or metal stent either of which may be coated with a suitable pharmaceutical substance or by rinsing the diseased tissue with medicaments, for example, by using a perfusion catheter.

However, above-mentioned systemic administration of pharmaceutical substances generally is achieved by oral administration or by an intra-arterial or intravenous injection. As a consequence, the pharmaceutical substances are released into the blood stream and are distributed not solely to the affected vessel or organ. Thus, merely small amounts of the therapeutic substances will arrive at the vessel segment or organ part which has to be treated. Especially for serious diseases, necessitating a high dose of medicaments to be administered to the diseased vessel or organ, systemic administration may cause harmful side effects since also surrounding healthy vessels and organs are contacted with the pharmaceutical substances. Thus, the effectiveness and dose of medicaments that can be administered is limited.

As mentioned, also balloon catheters and stents can be used to treat a stenosis. However, a mechanical expansion of restricted vessels and organs generally results in relapses of up to 60% of the treated area which will, thus, need a repeated treatment. The reasons for these relapses can be a recoil of the vessel or organ wall directly following treatment or inflammatory processes. A relapse may also be caused by a hyperproliferation of the neointima tissue induced by micro-lacerations of the tissue which occur at the site of mechanical expansion.

To counteract the mentioned recoil of the vessel or organ, metal stents may be implanted which dilate the vessel and organ lumens permanently. However, such stent implantation generally results, similarly to the treatment by balloon expansion, in micro-lacerations of the inner tissue layers which lead to the generation of excess scar tissue which is caused by the hyperproliferation of the neointima as mentioned above. As a consequence, a restenosis, i.e. a recurrent narrowing of the vessels and organs is observed for about 30% of the vessels and organs treated with a stent implantation.

To counteract the mentioned neointima hyperproliferation, anti-proliferative medicaments may be applied. Application of such cytostatic medicaments was attempted by directly coating balloon catheters and metal stents with suitable anti-proliferative pharmaceutical substances or by rinsing of the vessel and organ walls with such anti-proliferative substances.

In case of balloon catheters, the medicaments are generally directly applied to the surface of the balloon. This, however, frequently leads to a premature release of the medicaments from the balloon surface into the bloodstream before they can be brought into contact with the target vessel or organ since said medicaments generally exhibit a poor affinity to the surface of the balloon catheter. Especially hydrophilic substances are prone to be released prematurely from the balloon surface during positioning of the balloon at the area to be treated (wash-off effect).

Furthermore, the coating of balloon catheters with pharmaceutical substances frequently leads to local adhesions or encrustations at contact sites between balloon folds which prevents a homogeneous coating of the balloon catheter. Furthermore, due to the tight folding, a balloon catheter has a tendency to self-inflate which causes the medical substance to be stripped off when its surface contacts e.g. the transport protector of the balloon catheter or when it contacts a vessel wall prior to reaching the target site of application in the patient. As a consequence, in most cases, a significant and unpredictable amount of pharmaceutical substance is lost from the balloon catheter before the site of application is reached and, thus, only a reduced and undefined amount of the medicament will be brought into contact with the diseased vessel or organ tissue.

In case pharmaceutical substance coated metal stents are applied, the total surface of the struts of the stent will contact only about 15% of the vessel or organ wall when the stent is brought into its expanded state. Thus, only an insufficient area of affected tissue will be contacted and can be treated with the medicament. Furthermore, metal stents coated with pharmaceutical substances will generally reside permanently in the patient which, in the long run, tends to cause complications such as hypersensitivity, inflammation, and thromboses.

Attempts were also made to rinse the diseased vessel region or the diseased organ lumen using a perfusion catheter or a catheter having a double lumen. When using such catheters, however, the pharmaceutical substance is brought into contact with the affected tissue regions via diffusion. Therefore, a large amount of the medicament will not reach the diseased tissue but will be flushed and transported into downstream vessels and organs which leads to a significant exposure of these tissues to cytostatic substances, leading to harmful side effects.

Thus, there is a need for a medical device which can be manufactured with relatively small effort and costs and which allows a homogenous and reproducible localized application of any pharmaceutical substance to a target tissue without the risk that during manufacturing, sterilization, storage, transport or use the pharmaceutical substance either sticks to contact sites between balloon folds which may lead to jamming or a non-homogenous coating and without the risk of any premature release or stripping off of the medical substances from the medical device, e.g. a stent or balloon catheter as mentioned above.

### SUMMARY OF THE INVENTION

To solve above-mentioned problems and further problems associated with the prior art medical devices and methods, the present invention provides an expansible hollow part, having at least one opening, which consists of an elastic biocompatible material and which comprises at least one biologically active substance and, optionally, at least one matrix compound, wherein the expansible hollow part is porous and/or comprises micro-cavities in its surface.

The invention also provides a method of producing an expansible hollow part having at least one opening, which consists of an elastic biocompatible material and which comprises at least one biologically active substance and, optionally, at least one matrix compound, comprising the steps:
(a) expanding the expansible hollow part to at least 110% of its non expanded circumference, and,
(b) contacting the outer surface of the expansible hollow part with at least one biologically active substance and/or at least one matrix compound.
   Also comprised is an expansible hollow part producible by the method of the invention.

Also provided is a medical device covered at least partially by the expansible hollow part according to the invention.

Further provided is a kit-of-parts comprising at least one expansible hollow part according to the invention.

The invention provides further a use of an expansible hollow part according to the invention for the preparation of an enhanced balloon catheter for the treatment of a disease or a medical insufficiency selected from the group consisting of a stenosis, a restenosis, a stricture, a defective bypass craft, a thrombosis, a dissection, a tumor, a calcification, an arteriosclerosis, an inflammation, an autoimmune response, a necrosis, an injured anastomosis, a lesion, an allergy, a wart, a hyperproliferation, an infection, a scald, an edema, a coagulation, a cicatrization, a burn, a frostbite, and a lymphangitis.

In a further aspect the invention provides an expansible hollow part according to the invention for the use as a therapeutical device for the treatment of a disease or a medical insufficiency selected from the group consisting of stenosis, restenosis, a stricture, a defective bypass craft, a thrombosis, a dissection, a tumor, a calcification, an arteriosclerosis, an inflammation, an autoimmune response, a necrosis, an injured anastomosis, a lesion, an allergy, a wart, a hyperproliferation, an infection, a scald, an edema, a coagulation, a cicatrization, a burn, a frostbite, and a lymphangitis.

In a further aspect the invention provides a use of an expansible hollow part according to the invention for protecting a medical device or a biological tissue from mechanical stress, thermal stress, chemical stress and/or micro organisms.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The object of the present invention is to provide a means for the local delivery of drug/drug combinations from a medical device such as a stent, or a balloon catheter having the advantage that the drug/drug combinations can be applied without any loss of expensive medicaments and that the full surface of the medical device can be utilized, i.e. the medicament can be transferred are-wide from the medical device to the target tissue, thus providing an effective means to counteract e.g. neointimal hyperplasia, restenosis, inflammation and thrombosis.

It is one unexpected finding of the present invention, that an expansible hollow part according to the invention can efficiently be coated with a biologically active substance in such way that said expansible hollow part solves the aforementioned problems.

Thus, in a first aspect, the invention provides an expansible hollow part, having at least one opening, which consists of an elastic biocompatible material and which comprises at least one biologically active substance and, optionally, at least one matrix compound, wherein the expansible hollow part is porous and/or comprises micro-cavities in its surface.

An expansible hollow part of the invention can preferably have the shape of a tubular structure having either one or, more preferably, two open ends. Preferably, the expansible hollow part of the invention is a tube. Furthermore, it is preferred that the expansible hollow part of the invention is sterile and/or bioresorbable. Materials which are bioresorbable are known in the art.

In a preferred embodiment, the expansible hollow part of the invention is not a foil. In a further preferred embodiment, the expansible hollow part of the invention does not comprise gelatine. Most preferably, the hollow part has a wall thickness of smaller than 1 mm in its non-expanded state.

In a further aspect, the invention relates to an expansible hollow part, having at least one opening, which consists of an elastic biocompatible material and which comprises at least one biologically active substance, and, optionally, at least one matrix compound, wherein said expansible hollow part is characterized in that it has an inner diameter smaller than 3, 2.5, 2.0, 1.5, 1, 0.9, 0.8, 0.7, 0.6, 0.5, 0.4, 0.3, 0.2 or smaller than 0.1 cm in its non-expanded state. Preferably, the inner diameter is smaller than 1 cm in its non-expanded state when measured at its narrowest or, more preferably, at its broadest section.

Also provided is an expansible hollow part, having at least one opening, which consists of an elastic biocompatible material and which comprises at least one biologically active substance, and, optionally, at least one matrix compound, wherein said expansible hollow part has a wall strength of smaller than 2 mm, 1 mm, 0.9 mm, 0.8 mm, 0.7 mm, 0.6 mm, 0.5 mm, 0.4 mm, 0.3 mm, 0.2 mm or smaller than 0.1 mm in its non-expanded state. Most preferably, the wall strength is smaller than 1 mm.

As used herein “micro-cavity” refers to either a hole or a furrow such as a groove. The cross-section of said furrow can have any shape (see e.g. FIG. 16). If the micro-cavity is a hole, the hole is a pit that can also have any shape (see e.g. FIG. 16) but a micro-cavity that is a hole is not a perforation, i.e. not an opening connecting the outer and inner surface of the hollow part of the invention. Thus, if an expansible hollow part of the invention comprises micro-cavities these cavities according to the invention do not penetrate the material of the hollow part, e.g. to connect any outer surface with an inner surface of the material. This is advantageous since the cavities do not substantially weaken the material which is thus, resilient against mechanical stress and can undergo a substantial expansion without tear. For the same reason it is preferred that the surface of the expansible hollow part may preferably not comprise a plurality of perforations through which liquid can penetrate when the expansible hollow part of the invention is in its expanded state. It is, thus also preferred that the surface of the expansible hollow part of the invention is substantially impermeable to liquid and/or gas.

In one example, the expansible hollow part of the invention can be produced, as will be outlined below in greater detail, by dipping a dipping former into a dipping bath (see examples and FIG. 1). Preferably, the expansible hollow part of the invention is dried in between the dipping rounds through the use of fans, heaters, blowers or the like or by freeze drying or vacuum drying techniques or the like. Once the expansible hollow part is “dry”, the thickness of its wall may be determined utilizing any number of measuring techniques. Preferably, no force is exerted onto the expansible hollow part when measuring the wall strength, i.e. the wall thickness. If a greater wall strength is desired, the expansible hollow part may repeatedly be dipped and dried until the desired thickness is achieved. Preferably, the solvent part of the dipping solution or dipping emulsion is such, that upon successive dippings, the solvent part will not re-dissolve the dried material on the surfaces of the expansible hollow part. It is preferred that non-organic solvents are utilized for the preparation of the dipping solution or dipping suspension. Preferably, the material of the expansible hollow part is selected such that the average tickness of the wall of the material in its relaxed state is at least 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0 times larger than the wall thickness, i.e. wall strength of the material when the expansible hollow part is in the maximally expanded state (i.e. without incurring irreverible structural damage to the material). Preferably, the material of the expansible hollow part of the invention is an amorphous polymer.

As used herein “expansible” refers to the ability of the material of the expansible hollow part according to the invention to reversibly expand its surface when exposed to mechanical stress, i.e. a force causing deformation. Thus, the surface will return to its original, i.e. “relaxed” configuration, when the stress is removed. As used herein “relaxed” means in the absence of any external forces except the average atmospheric pressure present on earth at an altitude of between zero and 500 m above sea level. If in a preferred embodyment the expansible hollow part of the invention is a tubular structure, it is preferred that “expansible” means that the tubular structure can be reversibly expanded in its circumference. In a preferred embodiment, the expansible hollow part of the invention is expansible to at least 110%, 115%, 120%, 140%, 160%, 180%, 200%, 400%, 600%, 800%, 1000%, 1200%, 1400%, 1600%, 1800% or to at least 2000% of the circumference of its non-expanded state. Preferably the expansible hollow part according to the invention has an inner diameter smaller than 3, 2.5, 2.0, 1.5, 1, 0.9, 0.8, 0.7, 0.6, 0.5, 0.4, 0.3, 0.2 or smaller than 0.1 cm in its non-expanded state. Most preferably, the inner diameter is smaller than 1 cm in its non-expanded state when measured at its narrowest section.

It is further preferred that the expansible hollow part according to the invention is porous and/or comprises micro-cavities in its surface. As used herein, “porous” or “porosity” refers to the property of a material. If a material is “porous”, it comprises pores, i.e. volume of void space interspersed in the material. Preferably, the pores are only open towards the outer or inner surface of the material. Thus, it is preferred that the pores are not flow-through pores fluidly connecting an outer and inner surface of the material. Preferably, the material only exhibits porosity at its surface. It is further preferred that adjacent pores, in particular surface pores may form automatically, when using certain materials or may be generated by various drilling or ablation technologies known in the art. Preferably, the material of the hollow part according to the invention has a macro porosity, meso and/or micro porosity, wherein macro porosity refers to the presence of pores greater than or equal to 800 nm, 700 nm, 600 nm, 500 nm, 400 nm, 300 nm, 200 nm, 100 nm or greater than or equal to 50 nm in diameter, meso porosity refers to pores greater or equal than 2 nm and less than 50 nm in diameter and micro porosity refers to pores smaller than 2 nm in diameter. Most preferably, the expansible hollow part of the invention has a maximum pore size of 1 μm. Porosity can be measured without undue burden by measuring the pore diameters in a representative cross section of the material using microscopy, such as electron microscopy. Preferably, the porosity of the expansible hollow part according to the invention is such that it is permeable to ethanol. It is noted that the porosity as it is used herein referes to the porositiy of the expansible hollow part in its non-expanded, i.e. relaxed state. While it is considered that the entire material used to produce the expanded hollow part is porous, it is also possible that only a surface region shows the indicated macro, meso or micro porosity, preferably the outer surface.

As mentioned, the material of the expansible hollow part according to the invention preferably comprises micro-cavities in its surface. This means that the material surface is rough, having an average roughness height of at least 2 nm, 10 nm, 100 nm, 1 μm, 10 μm, 20 μm, 30 μm, 40 μm, 50 μm, 60 μm, 70 μm, 80 μm, 90 μm, or of at least 100 μm. Roughness height is the average altitude of surface irregularities of the material (see FIG. 6). The average roughness height of said material is preferably at least 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0 times smaller in the expanded state than in the relaxed state of the expansible hollow part according to the invention.

It is generally preferred that the pores are elongated and are more deep than wide. Thus, it is preferred that the pore size is smaller than the roughness height. A preferred surface porosity has pores of between 10 μm-40 μm. More preferably, the average surface porosity is between 10 μm-40 μm and the material surface has a an average roughness height of at least 10 μm, 12 μm, 14 μm, 16 μm, 18 μm or 20 μm. In another embodiment the average surface porosity is between 2 μm-40 μm and the hollow part comprises micro-cavities that have a maximal depth of 40 μm, 45 μm, 50 μm, 60 μm, 70 μm, 80 μm, 90 μm, 100 μm, 150 μm, 200 μm, 250 μm, 300 μm, or of 350 μm.

Preferably the wall thickness of the expansible hollow part of the invention is between 200 μm and 1000 μm and the hollow part comprises micro-cavities that have a maximal depth of 100 nm, 1 μm, 10 μm, 20 μm, 30 μm, 40 μm, 50 μm, 60 μm, 70 μm, 80 μm, 90 μm or 100 μm. It is also preferred that the expansible hollow part has a wall strength of smaller than 2 mm, 1 mm, 0.9 mm, 0.8 mm, 0.7 mm, 0.6 mm, 0.5 mm, 0.4 mm, 0.3 mm, 0.2 mm or smaller than 0.1 mm in its non-expanded state. In another preferred embodiment the ratio between the maximal depth of the micro-cavities comprised in the expansible hollow part of the invention and the thickness of the wall of the hollow part is 0.5:1, preferably between 0.01-0.4:1.

The micro-cavities in the surface of the expansible hollow part can be generated by e.g. blasting the surface with sand, glass beads, or water. Preferably the micro-cavities are cut utilizing laser or plasma eroding techniques. The pore size can be adjusted by, e.g. chosing a suitable material composition of the expansible hollow part of the invention. Detailed instructions can be found, for example in an article by Steve Jons et. al., “Porous latex composite membranes: fabrication and properties” published in the Journal of Membrane Science, volume 155, issue 1, 31 Mar. 1999, p. 79-99. To generate pores and/or micro-cavities, the material of the expansible hollow part of the invention, e.g. a latex composition, may also be mechanically foamed before casting, mechanically punctured, or temporary fillers may be placed in the material before it is cast which are dissolved or digested out subsequently.

The use of materials which have the above-described prefered porosity and/or micro-cavities, for the production of an expansible hollow part according to the invention leads to the surprising effect that when an expansible hollow part of the invention is coated in its expanded state with a biologically active substance, the biologically active substance will effectively enter the pores and/or microcavities of the expansible hollow part.

When an expansible hollow part according to the invention is relaxed after the coating process, it has been found that, unexpectedly, a significant amount of the biologically active substance will reside in the pores, preferably the surface pores, and/or microcavities of the hollow part (see also FIG. 7). The pores and/or microcavities protect the biologically active substance from being sloughed off or from being prematurely dissolved away from the expansible hollow part of the invention. Thus, for example, a balloon catheter covered at least partially by the expansible hollow part of the invention can be used for the local administration of drugs, agents or compounds. As will be explained below, also stents can be used in conjunction with an expansible hollow part of the invention to obtain improved therapeutic effects. Also higher tissue concentrations of the drugs, agents or compounds are achievable since hardly any biologically active substance is lost prematurely due to mechanical stress or solvent exposure. The biologically active substance is effectively released when the expansible hollow part of the invention is expanded at the target site and a diseased vessel or organ region is brought into contact with the expanded surface of the expansible hollow part of the invention. As only a very small amount of the biologically active substance is released into the blood stream, systemic toxicity is reduced and the therapeutic ratio (efficacy/toxicity) of the biologically active substance—preferably an anti-restenosis, anti-inflammatory or anti-thrombotic agent—is improved. Furthermore, due to the increased concentration of the biologically active substance that is achievable in the affected tissue, a single treatment may suffice with better patient compliance.

Accordingly, in one embodiment the at least one biologically active substance is located in said pores and/or in said micro-cavities. Preferably the at least one biologically active substance and said matrix compound is located in said pores and/or in said micro-cavities. Preferred is an expansible hollow part of the invention, wherein more than 10%, 20%, 30%, 40%, 50%, 60%, 70%, 80% or more than 90% of the biologically active substance and optionally of the matrix compound is located in the pores and/or in the micro-cavities of the outer surface of the expansible hollow part, preferably, when the expansible hollow part of the invention is at its relaxed state. In a particularly preferred embodiment, at least 80% or more preferably at least 90% of the total mass of said at least one biologically active substance is in said pores and/or in said micro-cavities and the rest is on the surface (see e.g. FIG. 7B) of the elastic biocompatible material of the inventive hollow part at its relaxed state. The respective amount located in the pores and/or micro-cavities can be increased by increasing the expansion of the expansible hollow part during application of the biologically active substance and/or increase the depth of the micro-cavities and/or number and size of the pores. One technical effect of this preferred embodiment is that the biologically active substance is better protected against mechanical and chemical stress when the expansible hollow part according to the invention is in its relaxed state.

It has been surprisingly shown that elongated micro-cavities show an advantageous feature that they are essentially closed if the expansible hollow part comprising such micro-cavities is in its non-expanded state and that they open up to a significant degree when the hollow part is expanded. This advantageous features is also shown in the examples and in FIGS. 9, 11, 14, 20 and 21. Thus, in a preferred embodiment of the expansible hollow part of the invention, the micro-cavities are elongated. Preferably, the elongated micro-cavities are selected from the group consisting of crescent-shaped furrows, sinuous furrows, circular furrows, elliptical furrows, furrows comprising one or more bends, straight furrows, bifurcated furrows and combinations thereof. In one preferred embodiment, the elongated micro-cavities have a length of not more than 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, or more than 6 mm. Irrespective of whether the micro-cavities are holes or furrows, it is preferred that their depth is between 5 μm and 500 μm, which allows good mechanical strength and good loading capacities of the biologically active substance which is as further detailed below preferably loaded into the micro-cavities. As already mentioned, it is preferred that the cavities do not penetrate the surface of the hollow part and, thus, the cavity depth is preferably smaller than the thickness of the wall of the expansible hollow part of the invention.

As shown in the examples and figures herein, elongated micro-cavities have the ability of opening and closing depending on the expansion of the hollow part. If the hollow part is in its relaxed form, i.e. non-expanded form the surface openings of the elongated micro-cavities are shut. Thus, it is preferred that the micro-cavities comprised in hollow part are substantially closed when the expansible hollow part is in its non-expanded state. “Substantially closed” means that the distance between two opposing fringes of the surface opening of a micro-cavity is smaller than 10%, 20% and more preferably less than 30% of the depth of that micro-cavity. A pharmaceutically active compound located in a “substantially closed” micro-cavity is eluted preferably about 1.2-, 1.3-, 1.5-, 2- or 3-times more quickly from the micro-cavity than the same amount of the same pharmaceutically active compound located in a micro-cavity that has “opened-up” upon expansion of the hollow part. An average skilled person knows how to determine such release kinetics and one preferred method is also provided in the examples below. Preferably, the micro-cavities open up when the expansible hollow part is expanded, preferably when its diameter is expanded to at least 1.25-fold of the diameter that the expansible hollow part has in its non-expanded state. “Open up” means that the distance between two opposing fringes of the surface opening of a micro-cavity increases.

It is also preferred that the micro-cavities of an expansible hollow part of the invention form fringes protruding from the surface of the expansible hollow part when the expansible hollow part of the invention is expanded. This is also shown in FIGS. 12, 13, 14 and 20.

It has been found that the expansible hollow part of the invention can be further optimized to deliver higher concentrations of biologically active substance to the target tissue when the elastic biocompatible material of the hollow part comprises tilted micro-cavities such as shown in e.g. FIGS. 18 and 19. The micro-cavities are, thus, preferably tilted with respect to the surface of the hollow part and more preferably tilted in direction of the longitudinal axis of the expansible hollow part.

The tilted micro-cavities can have any three-dimensional shape as long as they are tilted with respect to the surface, i.e. a line going through the deepest pit of the micro-cavity and the opening of the micro-cavity in an expansible hollow part that is non-expanded is not perpendicular to the surface of the hollow part, i.e. is not surface normal. The tilting prevents the premature loss of the biologically active substance due to mechanical stress (e.g. abrasion) and solvent exposure.

Thus, in a preferred embodiment the hollow part of the invention comprises tilted micro-cavities in its surface. The term “tilted” in this sense means that the volume of the micro-cavity, which preferably has the shape of a tube, is tilted at least 10°, 20°, 30°, 40°, at least 45° or more with respect to a surface normal running through the center of the opening of the micro-cavity. Preferably, the micro-cavities are tilted with respect to the longitudinal axis of the expansible hollow part as shown in e.g. FIGS. 18 and 19. If the therapeutic hollow part of the invention is used to be inserted into a body cavity, for example on a stent, it is most preferred that the micro-cavities point axially symmetrical into the direction in which the expansible hollow part will be inserted (see for example FIG. 9A). As described above, the tilted pores and/or micro-cavities prevent wash out and sloughing off of the biologically active substance when the inventive expansible hollow part is inserted into a body cavity such as a vein, artery, etc.

Two particularly preferred embodiments of the tilted micro-cavities are shown in FIGS. 18 and 19. The hollow part comprising micro-cavities shown in FIG. 18 is preferably inserted into the body cavity in the direction indicated in the figure. The alternative preferred embodiment shown in FIG. 19 is preferably inserted and transported to the target site in the body-cavity following one of the two directions as shown in the figure.

In one preferred embodiment, the surface of the expansible hollow part of the invention comprises at least two types of micro-cavities that differ in their shape and/or cavity depth. Preferred shapes are shown in FIG. 16. FIG. 17 illustrates a non-limiting example of this embodiment. In this embodiment, the release kinetic of the biologically active substance is controlled by the surface texture of the expansible hollow part. One advantage of this embodiment is that it allows e.g. a multiphase-substance release kinetic which is controlled by the degree of expansion of the hollow part. For example, in one preferred embodiment, the expansible hollow part of the invention comprises two biologically active substances, wherein one biologically active substance is located in a first type of micro-cavities and the second biologically active substance is located in a second type of micro-cavities and wherein the first and the second type of cavities differ in their cavity depth and/or shape, preferably the cavity depth of the second cavity type is about 10%, 20%, 50%, 60%, 80%, 90%, 100%, 250%, 300% or more deeper than the cavity depth of the first cavity type. This particular embodiment of the expansible hollow part of the invention is particularly suitable if two biologically active substances, i.e. two pharmaceutical substances are to be administered using the expansible hollow part of the invention and said pharmaceutical substances differ in their solubility and/or in their ability to be absorbed by the target tissue that is brought into contact with the biologically active substances. Thus, to prevent premature dissolving (e.g. in a body fluid) of a first biologically active substance that is more hydrophilic than a second biologically active substance, the first biologically active substance can selectively be filled into the deeper micro-cavities and the second biologically active substance can selectively be filled into the shallower cavities. Furthermore, it may be desirable to release two pharmaceutical substances at the target site with a temporal displacement. Thus, if two different drugs are located in two different types of micro-cavities wherein the mico-cavities differ in their cavity-depth, the drug that is located in the shallower micro-cavity type will be released earlier during expansion of the hollow part than the drug that is located in the deeper micro-cavity type. A skilled person is well aware of how to manufacture expansible hollow parts, preferably tubular expansible hollow parts that comprise at least two different types of micro-cavities wherein the types of micro-cavities differ in their cavity depth and wherein each type of micro-cavity is selectively filled with a respective biologically active substance. In one example, a surface pattern of different types of micro-cavities as described above can be created by laser ablation technology, using different laser power settings and/or exposure times as described also herein below. In one particularly preferred method, an expansible hollow part as described above is generated by first forming (for example using thermal (e.g. laser), mechanic (e.g. drilling) or chemical (e.g. etching) treatment) and filling the first type of micro-cavities and subsequently forming and filling the second type of micro-cavities. If a hydrophobic and a hydrophilic bioactive compound are used to fill the micro-cavities, the filling of the cavities can occur e.g. by dipping or spraying and the filling may also be carried out in one embodiment while the expansible hollow part is in its non-expanded state.

Thermal (e.g. laser, molding) or mechanic (e.g. drilling) treatment of the elastic biocompatible material can also be used to generate the tilted micro-cavities that have been described above. It has been shown herein that when using a laser, only very short pulses and energies should be used to form the micro-cavities (see also further information below and the examples). Thus, a skilled artisan is able to generate a texture consisting of specific types of micro-cavities on the surface of the expansible hollow part and he can subsequently fill the respective micro-cavities with different biologically active substances using e.g. a spraying or printing technique such as described in e.g. Berger et al., “Ultrasonic Liquid Atomization: Theory and Application” 2nd edition: Partrige Hill, 2006. 1-177.

When using a mechanical or thermal means for inserting micro-cavities in the surface of an expansible hollow part of the invention, the formation and filling of the micro-cavities with said at least one biologically active substance can optionally also be carried out when the hollow part is in its relaxed, i.e. non-expanded state. Preferably, the micro-cavities are shaped such that flip-top lids are formed which open upon expansion of the expansible hollow part of the invention. Flip-top lids can be created by forming tilted micro-cavities as described herein above.

A skilled person can, without undue burden determine how much of the biologically active substance is located in the pores and/or in the micro-cavities. In one example, microscopy, preferably electron microscopy is used to determine the average amount of biologically active substance which is located in the pores and/or in the micro-cavities versus the amount which adheres to the remaining surface (see FIG. 7). For this microscopy approach, a representative cross section through an expansible hollow part of the invention is used. Alternatively, the amount of biologically active material which is located in the pores and/or micro-cavities can be determined by measuring the dissolution of the biologically active substance. Thereby the skilled person compares what amount of the biologically active substance is dissolvable from an expanded hollow part versus a non-expanded hollow part in a fixed period of time. For example, if in one example, the amount of the biologically active substance which can be dissolved in a given period of time under identical experimental conditions (same temperature, same solvent, same area of surface exposed to solvent) from an expansible hollow part according to the invention in its relaxed state is 80% less than the amount that can be dissolved when the expansible hollow part is in its expanded state, then it is defined that 80% of the biologically active substance is located in the pores and/or in the micro-cavities. As used herein, “expanded state” refers to a state, wherein the expansible hollow part of the invention is expanded to at least 110%, 115%, 120%, 140%, 160%, 180%, 200%, 400%, 500%, 600%, 800%, 1000%, 1200%, 1400%, 1600%, 1800% or to at least 2000%, preferably at least 500% of its circumference of its non-expanded state. As used herein “circumference” refers to the largest possible circumference of the expansible hollow part. Most preferably, the circumference of the largest possible imaginary sphere is used which fits inside, i.e. into the lumen of the expansible hollow part of the invention. When determining what percentage of biologically active substance is located in said pores and/or in the micro-cavities it is preferred that two comparable copies of an expansible hollow part of the invention are submerged in a suitable solvent in a solvent bath for preferably 1, 2, 3, 4, 5, 6, 7, 8, 9, or 10 minutes. The solvent may be selected from the group consisting of distilled water, phosphate buffered saline, whole blood, blood serum and an organic solvent such as an alcohol, preferably ethanol. The most preferred assay conditions that can be used when determining the amount of biologically active substance that is located in the pores and/or in the micro-cavities comprises a time period of 5 minutes, room temperature and the comparison of a hollow part in its relaxed state with a hollow part which is expanded to between 400% and 600% and most preferably to about 500% of the circumference of its non-expanded (i.e. relaxed) state.

A further preferred embodiment of the invention is the expansible hollow part of the invention, wherein more than 10%, 20%, 30%, 40%, 50%, 60%, 70%, 80% or more than 90% or all of the biologically active substance is located in the pores and/or in the micro-cavities of the inner surface of the expansible hollow part. Such preferred embodiment is obtainable, as will also be described below, by everting the expansible hollow part after it has been coated with the biologically active substance. The skilled person can determine the amount of biologically active substance which is located in the pores and/or in the micro-cavities as described above. Expansible hollow parts which are coated on the inside are particularly useful for wrapping medical devices or a biological sample for storage and/or transport (see also description below and FIG. 4).

Another preferred embodiment of the invention is the expansible hollow part of the invention, wherein more than 10%, 20%, 30%, 40%, 50%, 60%, 70%, 80% or more than 90% of the biologically active substance is located in the pores and/or in the micro-cavities of the inner and outer surface of the expansible hollow part.

Also preferred is the expansible hollow part of the invention, wherein more than 10%, 20%, 30%, 40%, 50%, 60%, 70%, 80% or more than 90% or all of the first biologically active substance is located in the pores and/or in the micro-cavities of the inner surface of the expansible hollow part and wherein more than 50% and preferably more than the same lower boundary value as defined for the first biological substance above, of a second biologically active substance is located in the pores and/or in the micro-cavities of the outer surface of the expansible hollow part.

When the expansible hollow part of the invention comprises pores and/or micro-cavities then it is preferred that the depth of the micro-cavities and/or number and size of the pores is such that the expansible hollow part of the invention comprises at least 0.5, 0.6, 0.7, 0.8, 0.9, 1, 2, 3, 4, 5, or at least 6 μg of the respective biologically active substance per square millimetre surface in its non-expanded state. Preferably, it comprises at least 6 μg of the respective biologically active substance per square millimetre surface in its non-expanded state, wherein wherein more than 10%, 20%, 30%, 40%, 50%, 60%, 70%, 80% or more than 90% or all of the biologically active substance is located in the pores and/or in the micro-cavities of the expansible hollow part. In another embodiment, the hollow part of the invention comprises preferably at least 3 μg of the respective biologically active substance per square millimetre surface in its non-expanded state, wherein more than 10%, 20%, 30%, 40%, 50%, 60%, 70%, 80% or more than 90% or all of the biologically active substance is located in the pores and/or in the micro-cavities of said expansible hollow part. In another embodiment, the hollow part of the invention comprises preferably at least 3 μg of the respective biologically active substance per square millimetre surface in its non-expanded state, wherein more than 10%, 20%, 30%, 40%, 50%, 60%, 70%, 80% or more than 90% or all of the biologically active substance is located in the pores and/or in the micro-cavities of said expansible hollow part, wherein said pores and/or said micro-cavities are in the outer surface of the hollow part.

As described above, the expansible hollow part of the invention preferably comprises or consists of an amorphous polymer. In a more preferred embodiment, the elastic biocompatible material of the expansible hollow part according to the invention consists, comprises or essentially consists of a material selected from the group consisting of:

latex, polyvalerolactone, poly-s-decalactone, polylactic acid, polyglycol acid, polylactide, polyglycolide, co-polymer of polylactide and polyglycolide, poly-ε-caprolactone, polyhydroxy butyric acid, polyhydroxybutyrate, polyhydroxyvalerate, polyhydroxybutyrate-co-valerate, poly(1,4-dioxan-2,3-dione), poly(1,3-dioxan-2-one), poly-para-dioxanone, polyanhydride, polymaleicacidanhydride, polyhydroxymethacrylate, fibrin, polycyanoacrylate, polycaprolactondimethylacrylate, poly-β-maleic acid, polycaprolactonbutylacrylate, multiblockpolymers made of oligocaprolactondiole and oligodioxanondiole, polyetherestermultiblockpolymers made from PEG and polybutylenterephtalate, polypivotolactone, poly-glycolic acid trimethylcarbonate polycaprolactonglycolide, poly(g-ethylglutamate), poly(dth-iminocarbonate), poly(dte-co-dt-carbonat), poly(bisphenol A-iminocarbonate), polyorthoester, poly-glycolic acid-trimethylcarbonate, polytrimethylcarbonate polyiminocarbonate, poly(n-vinyl)-pyrrolidone, polyvinylalcohols, polyesteramide, glycolized polyester, polyphosphoester, polyphosphazene, poly(p-carboxyphenoxy)propane], polyhydroxypentanoic acid, polyethylenoxidpropylenoxid, polyurethane, polyurethane comprising amino acids, polyetherester like polyethyleneoxide, polyalkeneoxalate, lipids, carrageenane, fibrinogen, starch, collagene, protein-based polymers, polyaminoacids, zein, polyhydroxyalkanoate, pectic acid, actinic acid, carboxymethylsulfate, albumine, hyaluronic acid, chitosane, heparanesulfate, heparine, chondroitinsulfate, dextrane, β-cyclodextrine, copolymers comprising PEG and polypropyleneglycole, gummi arabicum, guar, gelatine, collagen-n-hydroxysuccinimide, phospholipids, polyacrylic acid, polyacrylate, polymethylmethacrylate, polybutylmethacrylate, polyacrylamide, polyacrylonitrile, polyamide, polyetheramide, polyethyleneamine, polyimide, polycarbonate, polycarbourethane, polyvinylketone, polyvinylhalogenide, polyvinylidenhalogenide, polyvinylether, polyisobutylene, aromatic compounds comprising a polyvinyl functional group, polyvinylester, polyvinylpyrollidone, polyoxymethylene, polytetramethyleneoxide, polyethylen, polypropylen, polytetrafluorethylen, polyetherurethane, silicon-polyetherurethane, silicon-polyurethane, silicon-polycarbonat-urethane, polyolefin-elastomers, epdm-rubber, fluorosilicone, carboxymethylchitosane, polyaryletheretherketone, polyetheretherketone, polyethylenterephtalate, polyvalerate, carboxymethylcellulose, cellulose, rayon, rayontriacetate, cellulosenitrate, celluloseacetate, hydroxyethylcellulose, cellulosebutyrate, celluloseacetatebutyrate, ethylvinylacetate, polysulfone, epoxy-resin, abs-resin, silicone like polysiloxane, polydimethylsiloxane, polyvinylhalogens, cellulose-ether, cellulose-triacetate, copolymers mixtures and derivatives thereof. As used herein, the term “derivative” refers to a chemical compound or molecule made from a parent compound or molecule by one or more chemical reactions. Preferably, however, the parent molecule must still be comprised in its structure in a “derivative”. Additionally, the derivative preferably has a molecular weight which is not greater than twice the molecular weight of the parent compound or parent monomer if the compound is comprised in a polymer.

In a more preferred embodiment, the elastic biocompatible material of the expansible hollow part according to the invention is selected from the group consisting of natural rubber, synthetic polyisoprene, a copolymer of isobutylene and isoprene, a halogenated butyl rubbers, a polybutadiene, a styrene-butadiene rubber, a copolymer of polybutadiene and acrylonitrile, a hydrogenated nitrile rubber, a chloroprene rubber, a polychloroprene, latex, a neoprene, a baypren. In another preferred embodyment the elastic biocompatible material is selected from a saturated rubber that cannot be cured by sulfur vulcanization such as ethylene propylene rubber, ethylene propylene diene rubber, epichlorohydrin rubber, polyacrylic rubber, silicone rubber, fluorosilicone rubber, fluoroelastomers, perfluoroelastomers, polyether block amides, chlorosulfonated polyethylene, ethylene-vinyl acetate, a resilin proteins and an elastin protein. Also either carbon black or silica may be added to the elastic biocompatible material of the expansible hollow part according to the invention up to a concentration of about 30 percent by volume which will raise the elastic modulus of the rubber material by a factor of about two to three.

In a most preferred embodiment, the elastic biocompatible material of the expansible hollow part according to the invention is latex, especially Guayule (Parthenium argentatum) latex which is hypoallergenic or isoprene. Latex is particularly prefered if the hollow part material has surface porosity since a suitable porosity is naturally formed when latex is used as material.

A further preferred embodiment of the invention is the expansible hollow part according to the invention, wherein the biologically active substance is selected from the group consisting of: a vasoconstrictor, a vasodilatator, a muscle relaxant, an antimycotic, a cytotoxic agent, a prodrug of a cytotoxic agent, a virustatic, a physiological or pharmacological inhibitor of mitogens, a cytostatic, a chemotherapeutic, an adrenocorticostatic, a β-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an angiogenesis inhibitor, an anticholinergic, an enzyme, a coenzyme, an antihistaminic, an antihypertensive, an antihypotensive, an anticoagulant, an antiseptic, an antihemorrhagic, a beta-receptor antagonist, a calcium channel antagonist, an antimyasthenic, an antiphlogistic, an antipyretic, a glucocorticoid, a haemostatic, an immunoglobuline or its fragment, a chemokine, a mitogen, a cell differentiation factor, a hormone, an immunosuppressant, an immunostimulant, a mineralcorticoid, a narcotic, a vector, a peptide, a (para)-sympathicomimetic, a (para)-sympatholytic, a protein, a cell, a sedating agent, a spasmolytic, a wound-healing substance, and combinations thereof.

In a more preferred embodiment, the biologically active substance of the expansible hollow part is selected from the group consisting of a RNA-oligonucleotide, a DNA-oligonucleotide, β-estradiol, 1,11-dimethoxycanthin-6-on, 12-beta-hydroxypregnadien 3,20-dion, 13,18-dehydro-6-alpha-senecioyloxychaparrin, 14-dehydroagrostistachin, 17-hydroxyagrostistachin, 1-hydroxy-11-methoxycanthin-6-on, 2-chloro-deoxyadenosin, 3-deazaadenosin, 4,7-oxycycloanisomelic acid, 4-hydroxyoxycyclophosphamid, abciximab, ace-inhibitors like captopril, acemetacin, acetylvismion B, aclarubicin, actinomycin, ademetionin, adriamycin, aescin, afromoson, agroskerin, agrostistachin, akagerin, aldesleukin, amidoron, aminoglutethemid, amsacrin, anakinra, anastrozol, anemonin, angiopeptin, anopterin, antimykotika, antiprozoale agentien like chloroquine, antisense oligonucleotide, antithrombin, antithrombotika, apocymarin, apoptosis inhibitors, apoptosis modulators like p65, argatroban, aristolactam-aii, Aristolochia Acid, ascomycin, asparaginase, aspirin, atorvastatin, auranofin, azathioprin, azelastin, azithromycin, baccatin, baccharinoide B1, B2, B3 and B7, bacitracin, bafilomycin, barringtogenol-C21-angelat, basiliximab, batimastat, bendamustin, benzocain, berberin, betamethason, betulin, betulinic acid, bevacizumat, bfgf-antagonisten, bilobol, biorest, bisparthenolidin, bivalirudin, bleomycin, b-myc-antisense, bombrestatin, bosentan, boswellic acid, bruceanole A, B and C, bruceantinoside C, bryophyllin A, busulfan, c myc specific antisense oligonucleotide, cadherine, camptothecin, capecitabin, carboplatin, carmustin, cefaclor, cefazolin, cefotixin tobramycin, celecoxib, cepharantin, cerivastatin, CETP-inhibitoren, cheliburinchlorid, chlorambucil, chloroquinphosphat, cictoxin, ciglitazone, cilazapril, cilostazol, ciprofloxacin, cisplatin, cladribin, clarithromycin, clotrimazol, colcemid, colchicin, concanamycin, coronarin A, B, C and D, coumadin, cox-2-Inhibitor, C-proteinase inhibitors, c-type natriuretic peptide (cnp), cudraisoflavon a, curcumin, cyclophosphamid, cyclosporin A, cymarin, cytarabin, cytochalasin A-E, cytokinine inhibitors, D-24851 (calbiochem), dacarbazin, daclizumab, dactinomycin, daphnoretin, dapson, daunorubicin, deoxypsorospermin, desacetylvismion A, desulfurated and n-reacetylated heparin (hemoparin®), dexamethason, diclofenac, dicloxacyllin, anti proliferative compounds, anti-cancer compounds, dihydronitidin, dihydrousambaraensin, disopyrimid, disulferam, docetaxel, doxorubicin, dunaimycin, effusantin A, eicosanoide, ellipticin, enalapril, endothelinantagonistenenoxoparin, epicatechingallat, epigallocatechingallat, epirubicin, epothilone A and B, erythromycin, estradiol, estradiolbenzoate, estradiolcypionate, estramustin, estriol, estron, etanercept, ethinylestradiol, etobosid, etoposid, everolimus, excisanin A and B, exemestan, faktor Xa-inhibitor antibody, filgrastim, flecainid, flucytosin, fludarabin, fludarabin-5′-dihydrogenphosphate, fluorouracil, fluroblastin, fluvastatin, folimycin, formestan, fosfestrol, a free nucleic acid, ganciclovir and zidovudin, gemcitabin, gentamycin, tissue-plasminogen-activator, ghalakinosid, ginkgol, ginkgolic acid, glykosid 1a, gpllb/llla-platelet membrane receptor, griseofulvin, guanidylcyclase-stimulator, inhibitor of metalloproteinase-1 and 2, halofuginon, helenalin, heparin, hirudin, histaminantagonisten, hydrocortison, hydroxyanopterin, hydroxycarbamid, hydroxychloroquin, hydroxyusambarin, ibuprofen, idarubicin, ifosfamid, igf-1, indanocine, indicin, indicin-n-oxid, indomethacin, inotodiol, interferon A, irinotecan, isobutyrylmallotochromanol, isodeoxyelephantopin, iso-lridogermanal. maytenfoliol, josamycin, justicidin A and B, a terpenoide like kamebakaurin and hippocaesculin, kamebaunin, ketoconazol, ketoprofen, carbonsuboxides (mcs) and macrocyclic oligomers thereof, lanograstim (r-hug-csf), lapachol, L-arginine, lariciresinol, larreatin, lasiocarpin, leflunomid, letrozol, leukamenin a and b, levomenthol, lidocain, liriodenin, liriodenin, lisinopril, lomustin, lonazolac, longikaurin b, losartan, lovastatin, lycoridicin, macrogol, malloterin, mallotochromanol, mansonin, maquirosid A, marchantin A, margetin, maytansin, medroxyprogesteron, mefenamin acid, mefloquin, melatonin, meloxicam, melphalan, mercaptopurin, methotrexat, methoxylariciresinol, methylsorbifolin, metronidazol, miconazol, midecamycin, miltefosin, mitomycin, mitoxanthrone, mitoxantron, mizoribine, mofebutazon, molgramostim (rhuGM-csf), molsidomin, monoclonal antibodies, m-prednisolon, mutamycin, mycophenolatmofetil, mycophenolic acid, myrtecain, naproxen, natriumaurothiomalat, steroids like inotodiol, NFkB, NF-kB or Bcl-xL-antisense-oligonukleotides, non-steroid compounds (nsaids) like fenoporten, nifedipin, nimustin, nitidinchlorid, nitroprusside, nocadazole, NO-donors like pentaerythrityltetranitrate and syndnoeimine, nonivamid, virus particles comprising oligonucleotides, nystatin, o-carbamoylphenoxyaceticacid, ovatodiolide, oxaceprol, oxacillin, oxaliplatin, oxoushinsunin, paclitaxel, 6-a-hydroxy-paclitaxel, pancratistatin, pcna ribozyme, pdgf-antagonists like triazolopyrimidin and seramin, pegasparase, peginterferon a-2b, penicillamin, penicilline like dicloxacillin, pentostatin, periplocosid a, antiviral compounds like phenylbutazon and acyclovir, pioglitazone, piroxicam, pitavastatin, plaminogen-activator inhibitor-1, plasminogen-activator inhibitor-2, podophyllotoxin, podophyllicacid-2-ethylhydrazid, polidocanol, PPACK, pravastatin, probucol, procainimid, procarbazin, prolylhydroxylase inhibitors, propafenon, prostacyclin, prostaglandin, protamin, protoanemonin, prourokinase, psycorubin, quinidin, quinin, rapamycin, regenilol, restenase, retinoic acid, r-hirudin, ricin a, rosiglitazone, rosuvastatin, roxithromycin, s 100 protein, sanguinarin, scopolectin, sculponeatin C, selectin (cytokinantagonist), serotoninblocker, shikonin, simvastatin, sinococulin A and B, sirolimus (rapamycin), smc-proliferation-lnhibitor-2w, s-nitrosoderivate, somatostatin, sotolol, sphatheliachromen, spiramycin, β-estradiol, β-lapachon, β-sitosterin, statine, staurosporin, stizophyllin, streblosid, streptokinase, strychnopentamin, strychnophyllin, sulfasalazin, sulfonamide, syringaresinol, tacrolimus, tamoxifen, taxamairin a and b, taxotere, teepolyphenole, teniposid, terbinafin, tetracyclin, tezosentan, thioguanin, thioproteaseinhibitoren, thiotepa, tocopherol tranilast, tomenphantopin A and B, topotecan, tranilast, transresveratrol, trapidil, tremozolomid, treosulfan, tretinoin, triamcinolon, triptolid, troleandomycin, tropfosfamid, tubeimosid, tyrosin-kinase-inhibitors like tyrphostine, umbelliferon, urokinase, ursol acid, usambarensin, usambarin, valsartan, vapiprost, vasodilators like dipyramidol, VEGF-inhibitors, verapamil, vinblastin, vincristin, vindesin, vinorelbin, vismion A and B, vitronectin-receptor antagonists, warfarin, antibiotika like cefadroxil, yadanzioside N and P, zeorin, mixtures and derivatives thereof.

In a further preferred embodiment, the expansible hollow part of the invention comprises at least 0.5, 0.6, 0.7, 0.8, 0.9, 1, 2, 3, 4, 5, or at least 6 μg of the biologically active substance per square millimetre surface in its non-expanded state, irrespective whether said expansible hollow part comprises pores and/or micro-cavities.

As mentioned, the expansible hollow part of the invention may optionally also comprise a matrix compound. This matrix compound is useful to e.g. improve the affinity and/or adhesiveness of the hollow part with respect to a biologically active substance. Thus, preferred is the expansible hollow part according to the invention, wherein the matrix compound is selected from a group consisting of polyvalerolactone, poly-c-decalactone, polylactic acid, polyglycolic acid, polylactide, polyglycolide, copolymers of polylactide and polyglycolide, poly-ε-caprolactone, polyhydroxylbutyric acid, polyhydroxybutyrate, polyhydroxyvalerate, polyhydroxybutyrate-co-valerate, poly(1,4-dioxane-2,3-dione), poly(1,3-dioxane-2-one), poly-para-dioxanone, polyanhydride, polymaleicacidanhydride, polyhydroxymethacrylate, fibrin, polycyanoacrylate, polycaprolactondimethylacrylate, poly-b-maleic acid polycaprolactonbutylacrylate, multi-block polymers of oligocaprolactondiole and oligodioxanonediole, polyetherestermultiblockpolymers made of peg und polybutylene terephthalate, polypivotolactone, polyglycolic acid trimethylcarbonate polycaprolactonglycolide, poly(g-ethylglutamate), poly(dth-lminocarbonate), poly(dte-co-dt-carbonate), poly(bisphenol a-iminocarbonate), polyorthoester, polyglycolic acid trimethylcarbonate, polytrimethylcarbonate, polyiminocarbonate, poly(n-vinyl)-pyrrolidone, polyvinylalcohol, polyesteramide, glycolated polyester, polyphosphoester, polyphosphazene, poly[p-carboxyphenoxy)propane], polyhydroxypentanoic acid, polyanhydride, polyethyleneoxidepropyleneoxide, polyurethane, polyurethane having amino acids in the backbone, polyetherester like polyethyleneoxide, polyalkeneoxalate, lipid, carrageenane, fibrinogen, starch, collagene, polymers comprising protein, protein, polyaminoacid, synthetic polyaminoacid, zein, polyhydroxyalkaneoate, pectic acid, actinic acid, carboxymethylsulfate, albumin, hyaluronic acid, chitosan und derivatives thereof, heparanesulfate and its derivatives, heparine, chondroitinsulfate, dextrane, β-cyclodextrine, copolymers of peg and polypropyleneglycol, gummi arabicum, guar, gelatine, collagen-n-hydroxysuccinimide, phospholipid, polyacrylic acid, polyacrylate, polymethyl methacrylate, polybutyl methacrylate, polyacrylamide, polyacrylonitrile, polyamide, polyetheramide, polyethyleneamine, polyimide, polycarbonate, polycarbourethane, polyvinylketone, polyvinylhalogenide, polyvinylidenhalogenide, polyvinylether, polyisobutylene, polyvinyl compounds, polyvinyl ester, polyvinylpyrollidone, polyoxymethylene, polytetramethylene oxid, polyethylene, polypropylene, polytetrafluoroethylene, polyetherurethane, silicone-polyetherurethane, silicone-polyurethane, silicone-polycarbonate-urethane, polyolefin, polyisobutylene, epdm-rubber, fluorosilicone, carboxymethylchitosane, polyaryletheretherketone, polyetheretherketone, polyethylene terephthalat, polyvalerate, carboxymethylcellulose, rayon, rayontriacetate, cellulose nitrate, cellulose acetate, hydroxyethylcellulose, cellulosebutyrate, celluloseacetatbutyrate, ethylvinylacetate, polysulfone, parylene, epoxy resin, abs-resin, silicone like polysiloxane, polydimethylsiloxane, polyvinylhalogene, cellulose ether, cellulose triacetate, chiosane, N,N-diethylnicotinamide, N-picolylnicotinamide, N-allylnicotinamide, sodium salicylate, 2-methacryloyloxyethyl phosphorylcholine, resorcinol, N,N-dimethylnicotinamide, N-methylnicotinamide, butylurea, pyrogallol, N-picolylacetamide, procaine HCl, nicotinamide, pyridine, 3-picolylamine, sodium ibuprofen, sodium xylenesulfonate, ethyl carbamate, 6-hydroxy-N,N-diethylnicotinamide, sodium p-toluenesulfonate, pyridoxal hydrochloride, 1-methyl-2-pyrrolidone, sodium benzoate, 2-Pyrrolidone, ethylurea, N,N-dimethylacetamide, N-methylacetamide, isoniazid, iopromide, a contrast dye, iobitridol, iohexyl, iomeprol, iopamidol, iopentol, iopromide, ioversol, ioxilan, iotrolan, iodixanol, ioxaglate, and combinations, derivatives and copolymers of combinations thereof. As mentioned the matrix compound can also be a contrast dye useful to visualize the expansible hollow part of the invention during a surgical procedure. Particularly preferred contrast dyes are iobitridol, iohexyl, iomeprol, iopamidol, iopentol, iopromide, ioversol, ioxilan, iotrolan, iodixanol, ioxaglate, and combinations thereof.

In the following, methods are provided which are useful to produce an expansible hollow part according to the invention.

Thus, in one aspect, the invention provides a method of producing an expansible hollow part having at least one opening, which consists of an elastic biocompatible material and which comprises at least one biologically active substance and, optionally, at least one matrix compound, comprising the steps:

(a) expanding the expansible hollow part to at least 101%, 103%, 105%, 106%, 108%, 110%, 115%, 120%, 130%, 140%, 150%, 160%, 170%, 180%, 190%, or to at least 200%, preferably at least 200% of its non expanded circumference, and,

(b) contacting the outer surface of the expansible hollow part with at least one biologically active substance and/or at least one matrix compound.

Preferably, the contacting in step (b) is carried out using a method selected from the group consisting of dipping, spraying and printing such as inkjet printing. In one preferred embodiment, step (b) is carried out at room temperature. The expansion in step (a) is preferably carried out using an expansible medical device (such as a stent or a balloon catheter) or a mechanical tool. An example for a suitable mechanical tool is provided in FIG. 5.

As mentioned, a preferred matrix compound is parylene, which is a polyxylylene polymer, preferably manufactured from [2.2]paracyclophane, that is useful to coat an expansible hollow part of the invention. Thus, it is preferred that the method of producing an expansible hollow part of the invention comprises a further step of chemical vapor deposition of parylene preferably at low pressure onto expansible hollow part of the invention. This preferred step produces a thin protective polymer coating and is preferably carried out after the bioactive compound has been applied, i.e. after step (b).

In a preferred embodiment, the method of the invention further comprises at least one additional step selected from the steps consisting of:
(c) relaxing the expansible hollow part so that its circumference is smaller than, preferably 1%, 3%, 5%, 10%, 20%, 30% smaller than the circumference of the expansible hollow part in step (a);
(d) before carrying out step (a), forming the expansible hollow part in a dipping bath comprising the elastic biocompatible material in liquefied form and optionally, a biologically active substance; and
(e) mechanically everting the expansible hollow part.
   In step (d) the expansible hollow part can be formed for example using a dipping bath that comprises the elastic biocompatible material in liquefied form and optionally, a biologically active substance (see also for example, FIG. 1 below). Alternatively, in step (d) instead of a dipping bath also injection-molding can be used. Micro-cavities can be formed in step (d). In this case, the dipping former is preferably covered with pins or elongated protuberances and the formed dipping mold will preferably be everted after it has solidified as also shown in FIG. 22. Particularly preferred molds will generate micro-cavities formed such as described herein. A tilted micro-cavity has preferably a depth of between 0.1 μm and 100 μm. Methods to form a hollow part using a dipping bath is known in the prior art. One example is shown in FIG. 1.

It is preferred that step (a) is carried out before step (b), i.e. that the expansible hollow part of the invention is coated with the biologically active substance when it is in its expanded state. Using this procedure, a larger amount of biologically active substance enters micro-cavities and/or pores on the hollow part, leading to several advantages which are described above.

In another preferred embodiment of the method according to the invention steps (a) and (b) or (b) and (c) are carried out simultaneously.

Also preferred is the method according to the invention, wherein
(i) step (e) is carried out before step (a) and/or after step (b) and/or after step (c);
(ii) steps (d), (a) and (b) are carried out in that order;
(iii) steps (d), (e), (a) and (b), are carried out in that order; or
(iv) steps (b), (e), (b) are carried out in that order, whereby in the first step (b) a different biologically active substance is used than the second step (b).

In an alternative embodiment of the method of the invention according to the invention comprising steps (a) and (b) as outlined above, the method further comprises at least one additional step selected from the steps consisting of:
(c) relaxing the expansible hollow part so that its circumference is smaller than the circumference of the expansible hollow part in step (a);
(d) cutting elongated micro-cavities into the elastic biocompatible material; and
(e) mechanically everting the expansible hollow part.

Also in this embodiment, steps (a) and (b) or (b) and (c) can be carried out simultaneously.

In this embodiment it is preferred that the steps are carried out in one of the following orders:
(i) step (e) is carried out before step (a) and/or after step (b) and/or after step (c);
(ii) steps (d), (a) and (b) are carried out in that order;
(iii) steps (a), (d) and (b) are carried out in that order;
(iv) steps (d), (e), (a) and (b), are carried out in that order; or
(v) steps (b), (e), (b) are carried out in that order, whereby in the first step (b) a different biologically active substance is used than the second step (b).

In the above preferred embodiments of the method of the invention micro-cavities can be formed in step (d) by thermal or mechanical treatment. For example, micro-cavities can be formed by exposing the surface of the material in step (d) to e.g. short laser pulses. The laser pulse preferably has a pulse duration of between 50 and 500 fs and a pulse energy of between 1 μJ and 20 μJ preferably of 3 μJ. These laser settings have been found by the inventors to prevent melting, distorting and rupturing of the expanded material.

In a further aspect, the invention provides an expansible hollow part producible by the method of the invention.

It is to be understood that the sequence of steps described in the preferred embodiment above includes further preferred embodiments, wherein additional steps selected from the steps (a) thorough (e) are carried out in one or in several instances in between the individual steps of the above indicated sequence of steps. For example, a preferred embodiment of feature (iv) above is a sequence of steps (a), (b), (e), (a), (b) which is carried out in that order.

Also provided is a medical device covered at least partially by the expansible hollow part according to the invention. In a preferred embodiment, the medical device is selected form the group consisting of a stent, a balloon catheter, a probe, a prosthesis, an endoscope, a pace maker, a heart defibrillator and a perfusion catheter. In preferred embodiments the stent is a stent prosthesis. Also preferred is a medical device according to the invention, wherein the balloon catheter is capable of emitting an electromagnetic radiation and/or a vibrational mechanical energy. Also preferred is a medical device according to the invention, wherein said expansible hollow part and said medical device are in contact but do not adhere to each other.

Preferably, a balloon catheter is covered by the expansible hollow part according to the invention. Thus, when the balloon catheter is inflated, the expansible hollow part of the invention is expanded and the biologically active substance contacts the vessel or organ wall. This allows an efficient transfer of the biologically active substance from the hollow part to the tissue. In an alternative preferred embodiment, a stent can be used which is placed between the balloon catheter and the expansible hollow part or, alternatively, over the expansible hollow part which covers the balloon catheter. Thus, preferred is a medical device according to the invention, wherein the medical device is a balloon catheter; and wherein the expansible hollow part is covered at least partially by a stent. Also preferred is a medical device according to the invention, wherein the medical device is a stent; and wherein the stent is on a balloon catheter. When stents are used in combination with an expansible hollow part of the invention they may themselves be coated with a therapeutic biologically active substance or not. In any case, the region of tissue that can be brought into contact with the biologically active substance will be significantly higher (up to 75% more surface) than when a stent is used in the absence of a hollow part. When the transfer of a given biologically active substance into the target tissue is inefficient, the contact time, i.e. the time period in which the surface of the expansible hollow part contacts the tissue, may be increased. However, when using regular balloon catheters, the contact time preferably does not exceed 1 minute when the affected vessel is located in the torso and does not exceed 10 minutes when the target tissue is in a distal body region, such as a leg. Otherwise the patient may suffer an acute heart failure. To further increase the contacting time, perfusion catheters can be used in combination with the expansible hollow part of the invention.

In one preferred embodiment, the balloon catheter of the medical device according to the invention comprises a hot balloon, a cold balloon, an occlusion balloon, a valvuloplasty-balloon and/or a protection device.

Most preferred is a medical device according to the invention, wherein the balloon catheter is a perfusion catheter which comprises a hot balloon or a cold balloon.

The preferred use of a hot balloon in combination with an expansible hollow part of the invention allows the practitioner to further increase the amount of biologically active substance which is transferred to the affected tissue. The hot balloon is preferably heated to a temperature of between 45° C. and 70° C. for the time period, when the expansible hollow part is in contact with the target tissue. This temporary heat shock will render the contacted tissue more permeable for the at least one biologically active substance which is comprised on the expansible hollow part of the invention. Thus, also the contacting times can be reduced when applying a heat shock, reducing the stress on the patient. Most biologically active substances tolerate a short exposure to a temperature between 45° C. and 70° C. A preferred biologically active substance which can be used with a hot balloon catheter is a mitotic inhibitor selected from the group consisting of a taxane such as paclitaxel or docetaxel; a vinca alkaloid such as vinblastine, vincristine, vindesine or vinorelbine; colchicine and derivatives thereof.

In another preferred embodiment, the invention provides a medical device according to the invention, wherein the balloon catheter and the expansible hollow part are connected to each other by at least one clamping piece. A clamping piece is any mechanical device tethering the balloon catheter to the expansible hollow part, for example a thread.

In a further preferred embodiment, the invention provides a medical device according to the invention, wherein the circumference of the medical device in its non-expanded state is greater than the inner circumference of the expansible hollow part in its non-expanded state.

The invention also provides in one aspect a kit-of-parts comprising at least one expansible hollow part according to the invention and at least one medical device, preferably a medical device selected from the group consisting of a stent, a balloon catheter, a probe, a prosthesis, an endoscope, a pace maker, a heart defibrillator and a perfusion catheter. In preferred embodiments, the stent is a stent prosthesis.

Also preferred is the kit-of-parts according to the invention, wherein the balloon catheter comprises a hot balloon, a cold balloon, an occlusion balloon, a valvuloplasty-balloon or a protection device.

Further preferred is the kit-of-parts according to the invention, wherein the balloon catheter is capable of emitting an electromagnetic radiation and/or a vibrational mechanical energy.

In a preferred embodiment of the kit-of-parts according to the invention, the circumference of the medical device in its non-expanded state is greater than the inner circumference of the expansible hollow part in its non-expanded state. This allows a tight-fit of the expansible hollow part onto the medical device.

In a further aspect, the invention provides the use of an expansible hollow part according to the invention for the preparation of an enhanced balloon catheter for the treatment of a disease or a medical insufficiency selected from the group consisting of a stenosis, a restenosis, a stricture, a defective bypass craft, a thrombosis, a dissection, a tumor, a calcification, an arteriosclerosis, an inflammation, an autoimmune response, a necrosis, an injured anastomosis, a lesion, an allergy, a wart, a hyperproliferation, an infection, a scald, an edema, a coagulation, a cicatrization, a burn, a frostbite and a lymphangitis. In a further preferred embodiment of the use according to the invention, the disease or medical insufficiency may be a bone injury.

In another aspect, the invention provides an expansible hollow part according to the invention for the use as a therapeutical device for the treatment of a disease or a medical insufficiency selected from the group consisting of stenosis, restenosis, a stricture, a defective bypass craft, a thrombosis, a dissection, a tumor, a calcification, an arteriosclerosis, an inflammation, an autoimmune response, a necrosis, an injured anastomosis, a lesion, an allergy, a wart, a hyperproliferation, an infection, a scald, an edema, a coagulation, a cicatrization, a burn, a frostbite and a lymphangitis. In a further preferred embodiment, the expansible hollow part according to the invention may also be used as a therapeutical device for the treatment of a bone injury.

In preferred embodiments of the aforementioned medical use or expansible hollow part of the invention, the stenosis is selected from the group consisting of pyloric stenosis, biliary tract stenosis, phimosis, hydrocephalus, stenosing tenosynovitis, spinal stenosis and subglottic stenosis.

The invention also refers to the use of an expansible hollow part according to the invention for protecting a medical device or a biological tissue from mechanical stress, thermal stress, chemical stress and/or micro organisms. Preferred medical devices that can be protected are selected from the group consisting of a stent, a balloon catheter, a probe, a prosthesis, an endoscope, a pace maker, a heart defibrillator and a perfusion catheter. A preferred biological tissue that can be conserved and/or protected using the expansible hollow part of the invention is an organ, wherein the organ is preferably not in a living human being.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following figures and examples are merely illustrative of the present invention and should not be construed to limit the scope of the invention as indicated by the appended claims in any way.

### BRIEF DESCRIPTION OF THE FIGURES

FIG. 1: Example for a method of producing an expansible hollow part according to the invention. One or several dipping formers (DF) are dipped at least partially into a dipping bath (DB) comprising the elastic biocompatible material in liquefied form. The dipping former may have any shape and can, thus, also be, for example, a medical device.

FIG. 2: Example of an expansible hollow part of the invention, wherein the expansible hollow part is a drug cover which covers a balloon catheter. Such shaped expansible hollow part which is coated with at least one biologically active substance is preferably trimmed after production on both ends and tightly fitted around a medical device such as a balloon catheter. In this figure the biologically active substance is on the outside such that upon expansion of the balloon, the vessel wall (not shown) is contacted with the biologically active substance.

FIG. 3: Example of an expansible hollow part of the invention, wherein the expansible hollow part is used on an embolic protection device. The at least one biologically active substance is located in the pores and/or in the micro-cavities of the outer surface of the expansible hollow part which covers the embolic protection device. Thus, the biologically active substance is transferred from the expansible hollow part to the vessel cells (i.e., the “landing zone”) contacted by the embolic protection device.

FIG. 4 Example of an expansible hollow part of the invention, wherein the expansible hollow part is used for protecting a medical device, for example a hip implant. Preferably, the expansible hollow part comprises at least one biologically active substance on the inner surface of the expansible hollow part. Expansible hollow parts which are coated on the inside can be obtained, for example, by everting the expansible hollow part after coating it with the at least one biologically active substance (for example after step 2 shown in FIG. 5). When medical devices are stored and/or transported in a protective expansible hollow part as shown, the biologically active substance will be transferred from the expansible hollow part onto the medical device. After shipping and upon use, the expansible hollow part is optionally removed from the medical device which will be covered by the biologically active substance.

FIG. 5 Schematics of a coating process is shown in step 1 and 2. Schematics of mounting the expansible hollow part (in this figure also called “drug cover”) onto a medical device is shown in steps 3 and 4. In step 1, the expansible hollow part (in this figure also called “drug cover”—see (2)) is mounted on an expansion tool (3). Upon expansion, the hollow part is coated (4), for example on the outside, with at least one biologically active substance (step 2). If the expansible hollow part is to be mounted on a medical device, for example a balloon catheter and preferably a PTCA balloon (5), the medical device is inserted into the expansion tool as shown (step 3). Next, the expansion tool is retracted which allows the expansible hollow part to shrink onto the medical device (steps 3 and 4).

FIG. 5B Shown is a folded surface of a conventional balloon catheter without (left) and with (right) an expansible hollow part of the invention.

FIG. 6 Determining surface roughness. The average roughness height of the material surface is measured, e.g. by microscopy.

FIG. 7 The amount of biologically active material which is located in the pores and/or micro-cavities can be determined by e.g. microscopy. A cross section of a region close to the surface of an expansible hollow part of the invention is shown. “A” designates biologically active substance which is located in one exemplified pore/micro-cavity and “B” designates biologically active substance which is located on the surface of the expansible hollow part of the invention but which is not in pores and/or micro-cavities. The biologically active substance can more easily be dissolved from the hollow part when it is in its expanded state (right). This figure is a schematic illustration only and, thus, additional pores and/or micro cavities that may be present in an embodiment of the expansible hollow part of the invention are not shown.

FIG. 8 Without being bound by theory, an expansible hollow part when expanded over a tubular medical device such as an angioplasty balloon is subject to two major expansionary forces as depicted in FIG. 8. As the circumference of the expansible tube (2) increases there is an expansionary force on the surface (A). This force is acting into two directions, separated by an expansion line depicted as {Z}. A second force is exerted radial on the expansible tube (B) as the tubular medical device is expanded beneath the expansible tube.

FIG. 9 The interaction of the forces described in FIG. 8 leads to a non-circular expansion of all patterns and formations on the surface of the expansible tube. As depicted in FIG. 9 there will be little expansion in the direction of the dashed expansion-line while there is substantial expansion in a right angle to the expansion-line. Crescent (a) and circular (b) micro-cavities in form of furrows cut into the surface will open in a crescent-like shape as shown in FIGS. 9a and 9b, when the expansible hollow part is expanded (right panels).

FIG. 10 Preferred embodiments of elongated micro-cavities that can be used with the hollow part of the invention are shown. As shown in FIG. 10 elongated micro-cavities that include cuts/furrows that run essentially not perpendicular to the longitudinal axis of the hollow part of the invention such as e.g. single angle (P1), multiple angle furrows (P2), semi-ellipse or semi-circle (P3) as well as elongated micro-cavities having a shape as shown in (P4 to P6) can be used. These shapes exploit the described expansion characteristics to enhance the drug loading and shielding capacity of the corresponding three-dimensional cavities that are formed when the described patterns are cut into the surface of the expansible hollow part (here: tube) without cutting all the way through.

FIG. 11 FIG. 11 shows cavities that can be created when cutting into the surface of an expansible hollow part of the invention. When the expansible hollow part is in its expanded state, the elongated micro-cavities having a shape as shown in FIGS. 10 (P1, P2, P3 and P5) can give raise to cavities shown here as C1, C2, C3 and C5.

FIG. 12 If the expansible hollow part is tubular-shaped and, thus, has a curved surface, elongated micro-cavities located in the surface of the expansible hollow part may form protruding fringes when the expansible hollow part of the invention is expanded. FIG. 12 a) non-expanded, b) expanded mode) shows the cross-sectional effect that of the radial force (B) can exert on the expansible hollow part which is a tube in this example (2). Since the elongated micro-cavities are bent in their longitudinal axis, the inner curved part pivots upwards (right of the cavity) while the outer part pivots sideways and downwards (left side of the cavity). This also leads to a rotational movement of the cavities' bottom (market with a black square) which facilitates the expulsion of the biologically active compound out of the expanded cavity.

FIG. 13 The expansible hollow part of the invention may comprise tilted elongated micro-cavities as shown in this figure. This will further enhances the drug shielding capacity of the micro-cavity—here shown in (a) non-expanded and (b) expanded mode.

FIG. 14 An example for micro-cavities showing protruding fringes. The 3D illustration depicts also the superficial (A) and radial forces (B) exerted on curved cavities upon expansion. FIG. 14a shows the expansible hollow part which is in this example a tube (2) in its non-expanded mode featuring elongated micro-cavities having a crescent shape. Expansion of the hollow part causes the cavities (C3) to open and the fringes to protrude (lifted inner semi-cylindrical part). This facilitates expulsion of the biologically active compound and increases the mechanical friction of the expansible hollow part with respect to the tissue, improving its local fixation at the treatment site.

FIG. 15 Preferred embodiments of elongated micro-cavities are shown. The longitudinal axis of the expansible hollow part of the invention is indicated at the bottom of the figure. The shapes of preferred elongated micro-cavities shown as (a) through (f) and parts thereof can also be combined with each other in any order and the shapes shown may also be rotated in any direction with respect to the longitudinal axis.

FIG. 16 Four particularly preferred embodiments of the micro-cavities used in the invention are shown in cross-sectional view. As described herein, the micro-cavities used according to the invention can be holes and/or furrows. If the micro-cavities are furrows, then the cross-sections shown in (a) through (d) run substantially perpendicular to the longitudinal axis of the furrow-shaped elongated micro-cavities.

FIG. 17 A cross-section of a region close to the surface of an expansible hollow part of the invention is shown, where the surface of the hollow part comprises two different types of micro-cavities. The biologically active compound comprised in micro-cavity type I is exposed to the surface earlier during expansion (“intermediate state”) than the biologically active compound comprised in micro-cavity type II (“fully expanded state”), allowing in this example a biphasic release kinetic. “A” designates the biologically active substance which is located in exemplified micro-cavities. Also in context of this figure, the micro-cavities may be holes and/or furrows. If the micro-cavities are furrows, then the cross-sections shown in (a) through (d) run substantially perpendicular to the longitudinal axis of the furrow-shaped elongated micro-cavities.

FIG. 18 Examples of micro-cavities are shown that are tilted in a preferred direction. A longitudinal cross section of a region close to the surface of a non-expanded expansible hollow part of the invention is shown. In this preferred example, the micro-cavities are tilted away from the direction of insertion of the hollow part. This preferred embodiment further improves the protection of the biologically active substance from abrasion and wipe-off when the expansible hollow part of the invention is inserted into a body cavity such as an artery or a vein. This embodiment of the hollow part is preferably inserted in the direction of the arrow as shown. “A” designates biologically active substance which is located in the micro-cavities. The indicated micro-cavities may be holes and/or furrows. The distance between individual micro-cavities may be constant as shown in this figure, or random or having any other arrangement.

FIG. 19 A longitudinal cross section of a region close to the surface of a non-expanded expansible hollow part of the invention is shown. This figure illustrates a further preferred embodiment of the hollow part of the invention which protects the biologically active substance in the micro-cavities against abrasion and wipe-off upon insertion. The possible directions of insertion of the hollow part for this example are shown. “A” designates biologically active substance which is located in the micro-cavities. The indicated micro-cavities may be holes and/or furrows. The distance between individual micro-cavities may be constant as shown in this figure, or random or having any other arrangement.

FIG. 20 Images of an embodiment of the expansible hollow part of the invention taken with a scanning electron microscope. The images show half-circle shaped micro-cavities in the surface of an expanded hollow part of the invention, wherein the half-circle shaped micro-cavities have a diameter of 300 μm, a depth of about 120 μM and a width (opening) of about 100 μm. Thus, the micro-cavities are open when the hollow part is expanded, as also shown in the drawing in FIG. 14(b). The results shown were obtained for hollow parts consisting of polyisoprene. Similar results were obtained for hollow parts made from latex.

FIG. 21 Images of an embodiment of the expansible hollow part of the invention taken with a scanning electron microscope. The images show the half-circle shaped micro-cavities depicted in FIG. 20 in the surface of a non-expanded hollow part of the invention. As shown, the micro-cavities close tightly (as also illustrated in FIG. 14(a)) when the expansible hollow part of the invention is in its relaxed state.

FIG. 22 Shown are (a) unmodified dipping forms (1) and resulting expansible tubes (2); (b) a modified dipping form; (c) a modified expansible tube and (d) a modified expansible tube which has been everted.

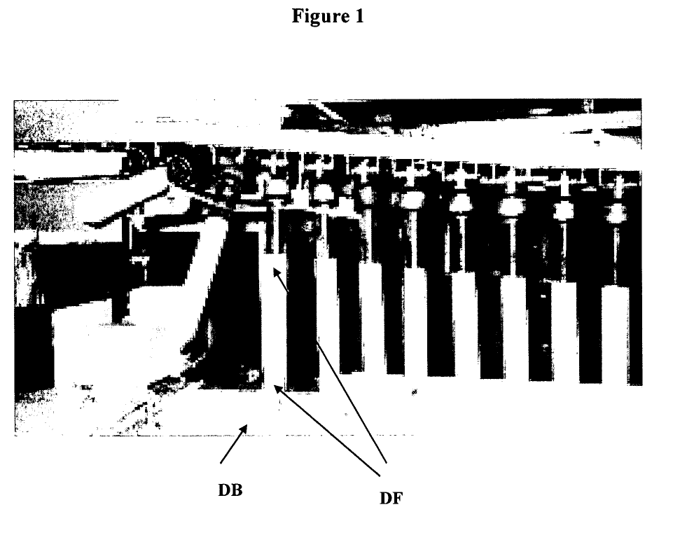

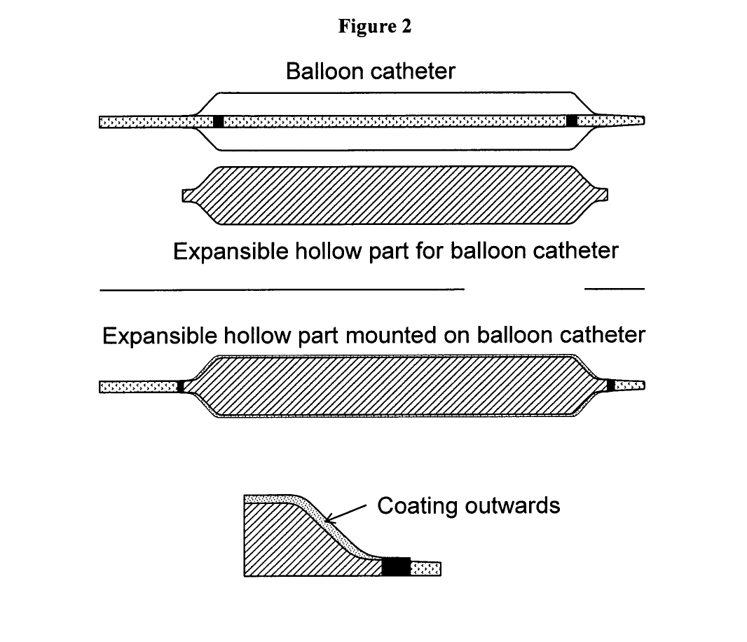

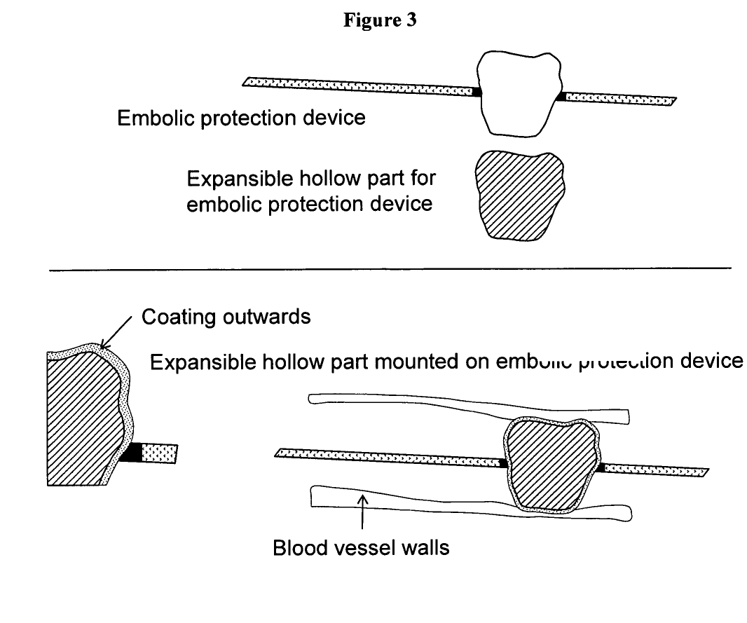

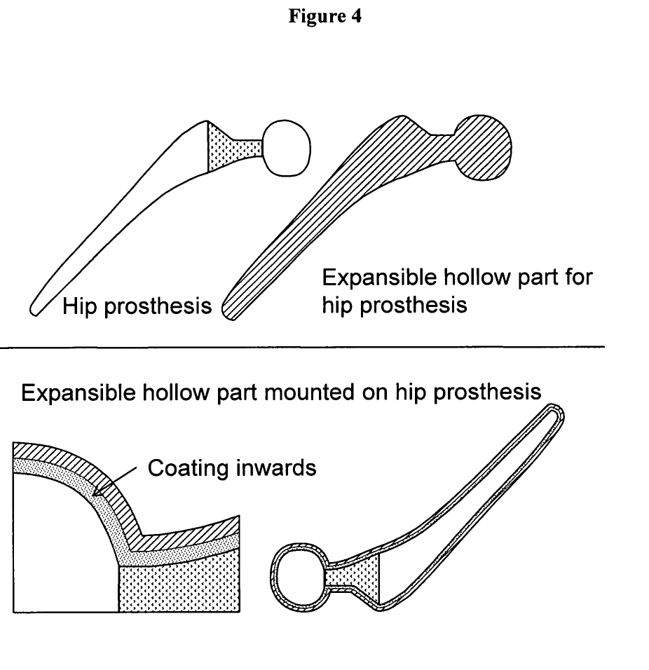

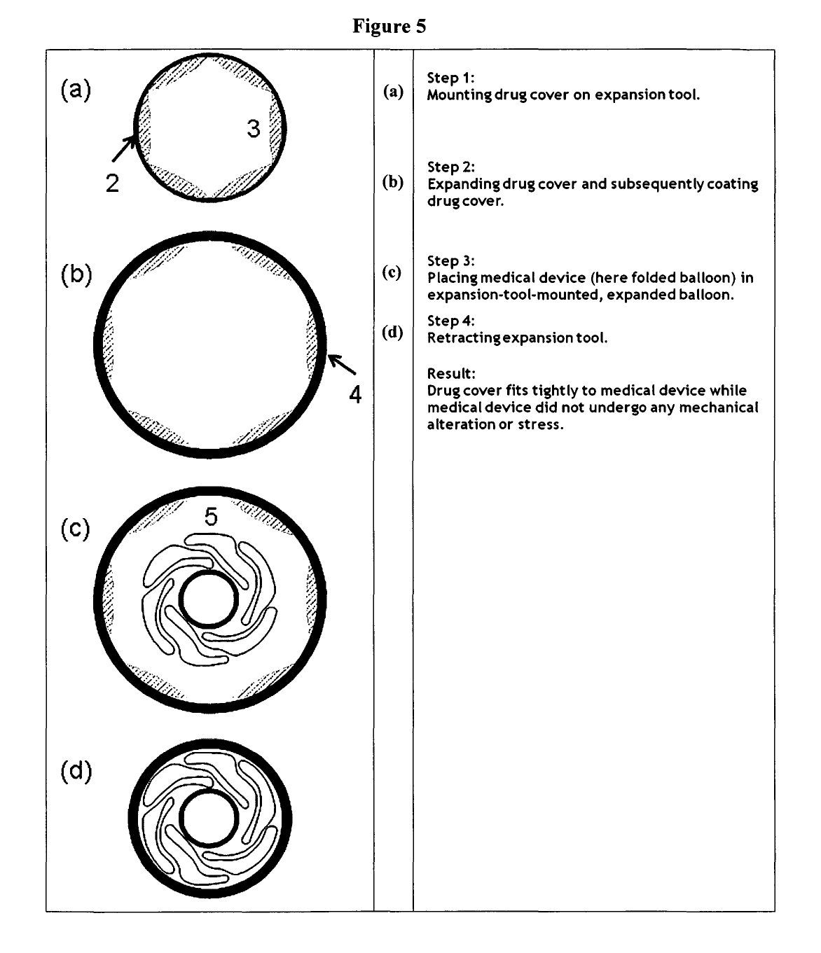

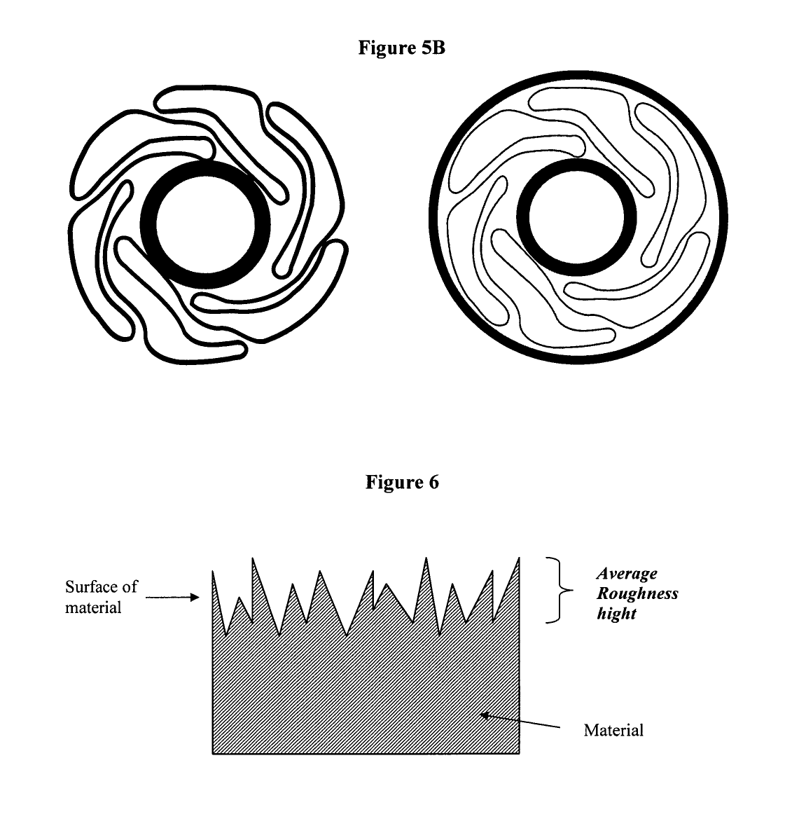

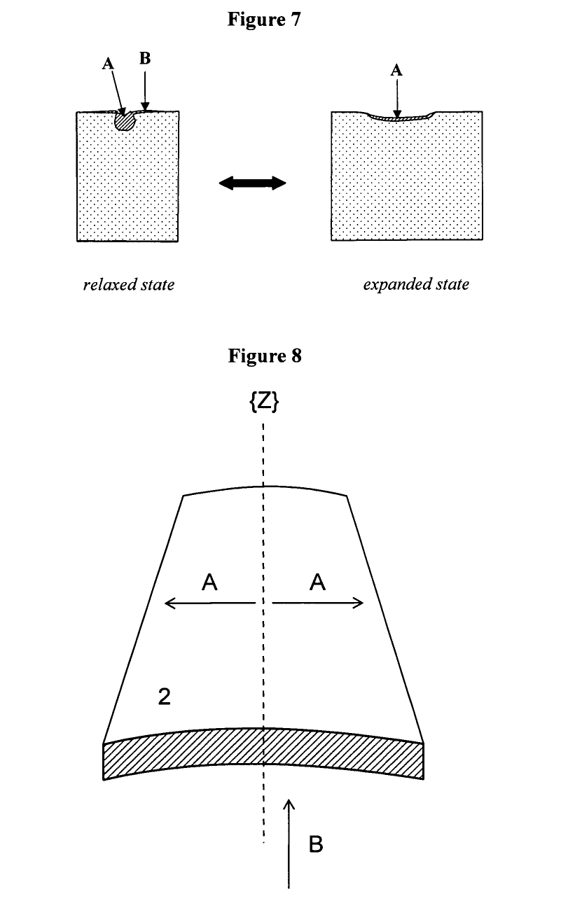

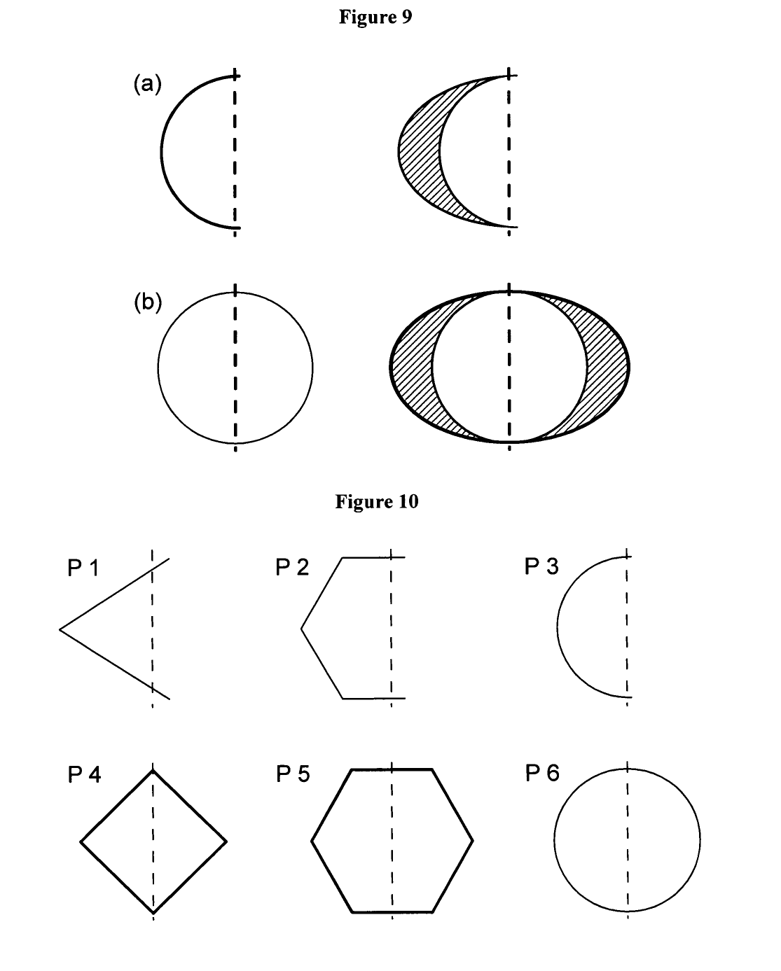

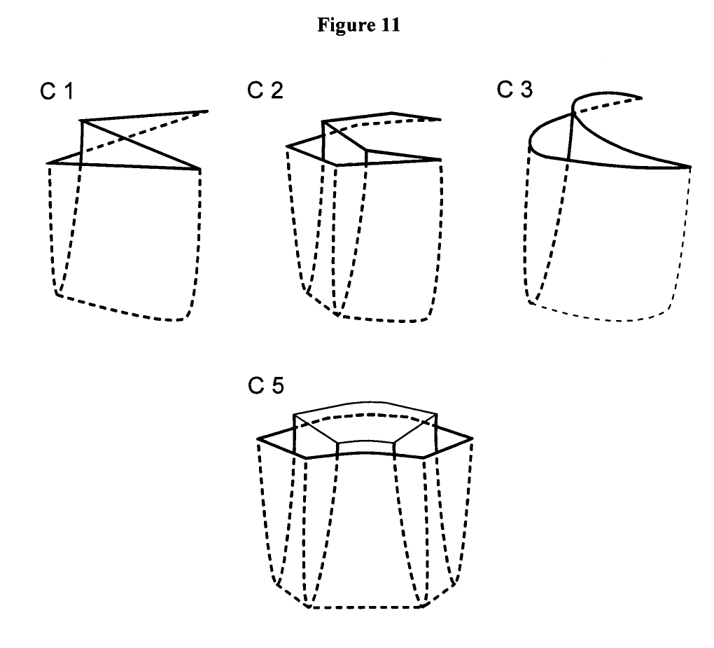

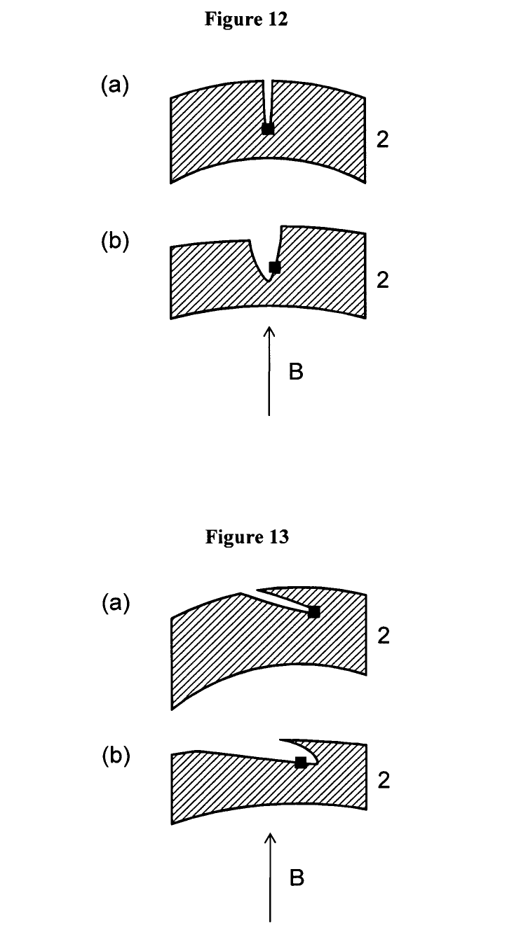

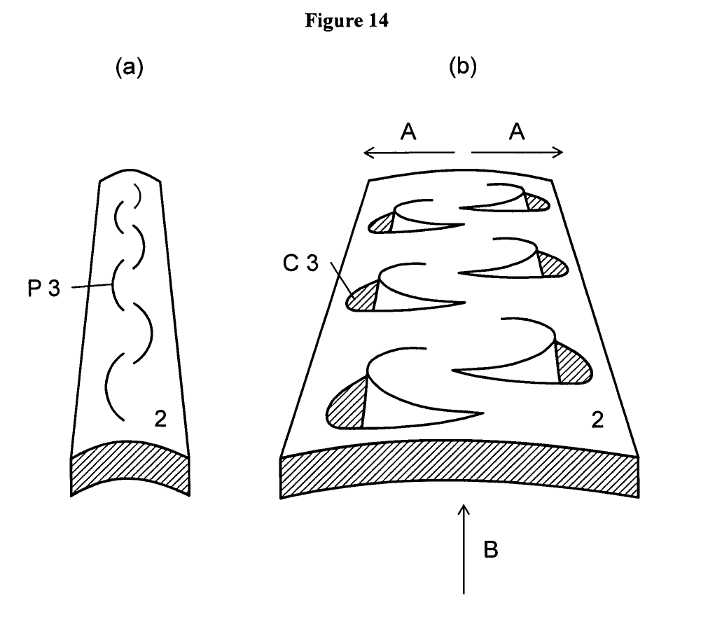

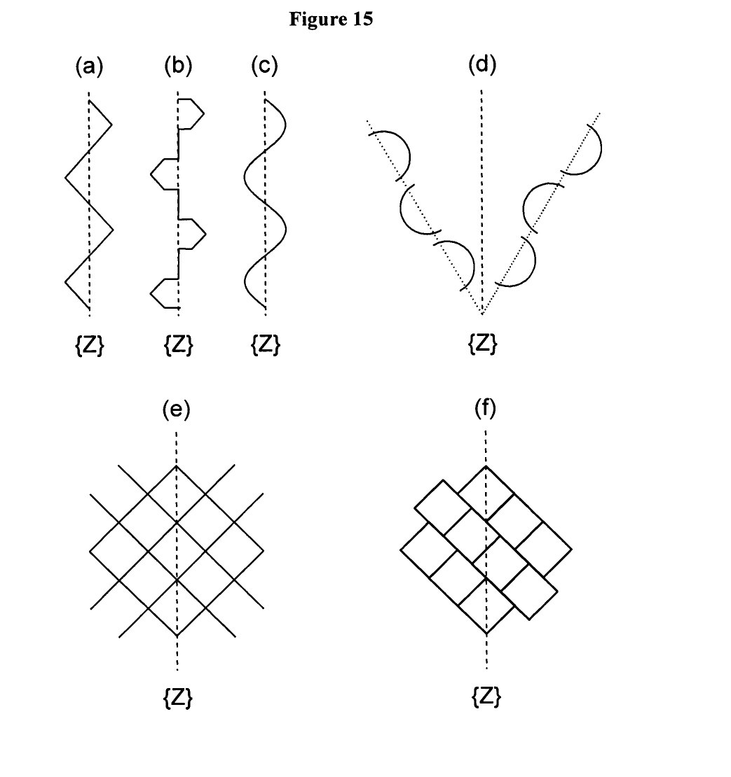

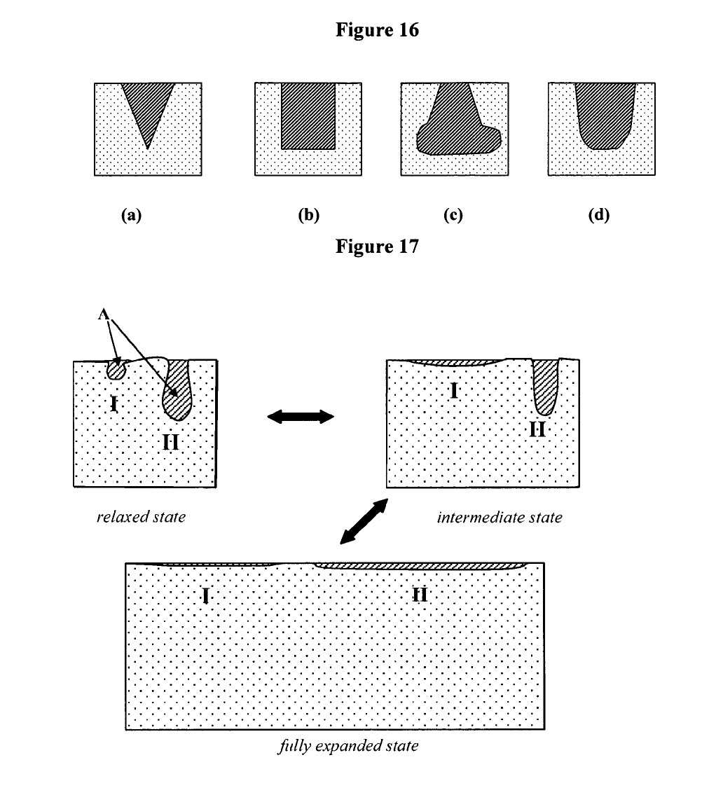

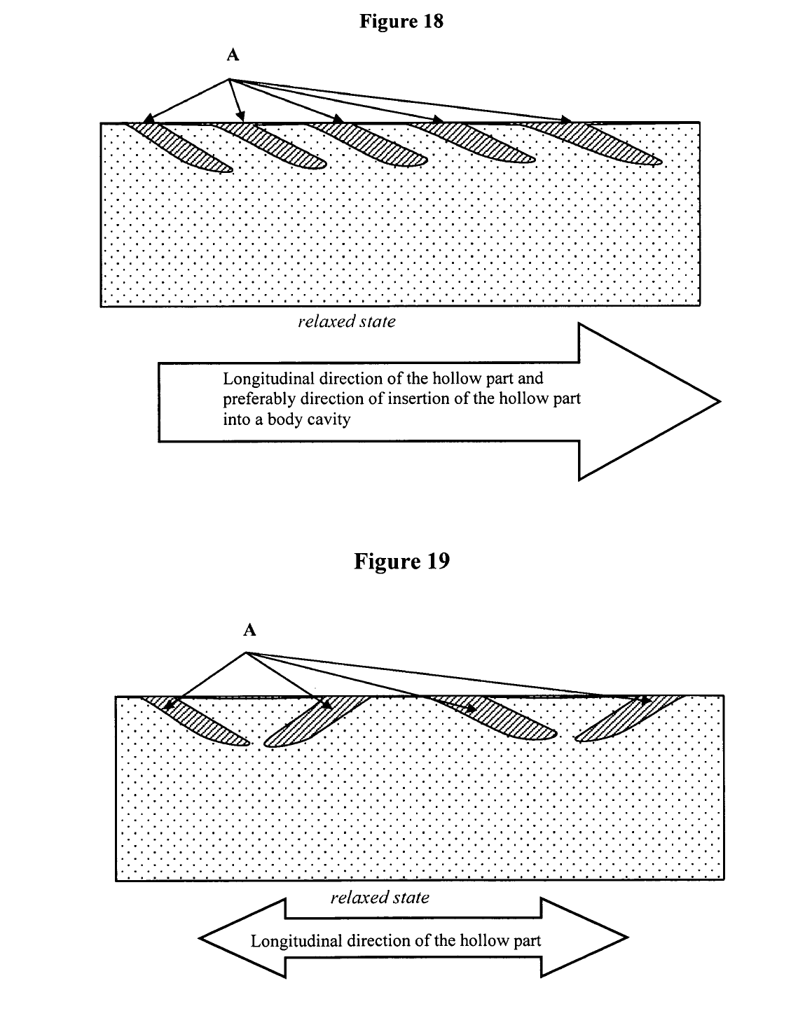

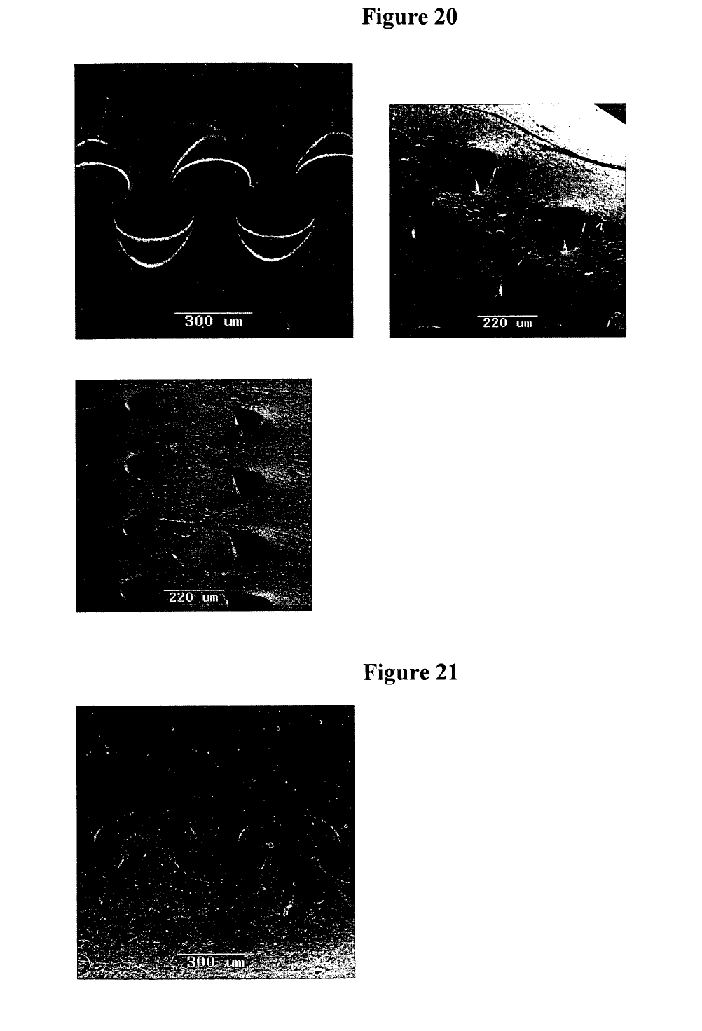

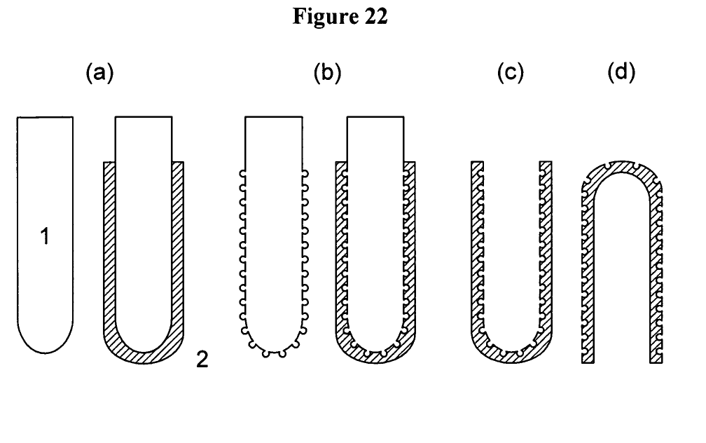

### EXAMPLES

### Example 1

Experimental data show that short-term application of paclitaxel to human endothelial progenitor cells (EPC) and smooth muscle cells (SCM) leads to apoptosis and therefore inhibition of cell proliferation and migration. This effect is dose and time dependent; rising concentrations in paclitaxel and longer application show higher inhibition of cell proliferation and migration. The expansible hollow part was shown to hold more than 6 μg/mm² paclitaxel in its non-expanded state.

The use of an expansible hollow part which comprised paclitaxel in an amount of between 1-6 μg/mm² has proven to be more effective in animal trials.

Thus, a most preferred application of the invention is therefore a paclitaxel-eluting expansible hollow part mounted on a balloon-catheter for the treatment of stenoses or restenoses. A further most preferred application is a metal stent mounted on a paclitaxel-eluting expansible hollow part mounted on a balloon-catheter for the treatment of stenoses or restenoses.

### Example 2

A tube-shaped expansible hollow part of the invention made from polyisoprene was expanded by a factor of 1.25 using an expansion device. Crescent shaped micro-cavities having an average length of about 300 μm were cut into the surface of the expanded hollow part using a titanium:saphire-laser. The laser settings were as follows: pulse duration: 150 fs; wavelength: 800 nm; spot-size about 30 μm; pulse energy 3 μJ.

### Example 3

A tube-shaped expansible hollow part made from polyisoprene featuring crescent shaped micro-cavities as described in example 2 was expanded by a factor of 2.5 using an expansion device. Subsequently the hollow part was placed in an ultrasonic spray system that uses high-frequency sound waves to produce a fine spray of liquid.

The operating vibration frequency of the spraying nozzle was set at 120 kHZ to produce a median drop diameter of less than 20 μm from a trapidil ethanol solution of a concentration of 250 mg/ml.

The expansible hollow part mounted on the expansion tool was placed under the spraying nozzle and transported longitudinal through the spraying gas at a speed of 1 mm/sec while constantly being rotated at 60 rpm.

After three consecutive runs the coating layer of the hollow part was examined using an optical microscope. Over 95% of the crescent shaped micro-cavities exhibited a total filling state while less than 5% exhibited a sub-total filling state defined as the appearance of funnels or bridging over the fringes of the micro-cavities.

### Example 4

Using the method described in example 3 a sample of expansible hollow parts without any surface modification and a sample of expansible hollow parts with described crescent shaped micro-cavities where coated with an equal amount of trapidil (N,N-diethyl-5-methyl-[1,2,4]triazolo[1,5-a]pyrimidin-7-amine) to obtain a drug loading of about 6 μg/mm².

The hollow parts where subsequently mounted on balloon catheters and placed into a plastic tubing in which 0.9% sodium chloride solution with a temperature of 37° C. was circulating. The hollow parts remained in the tubes while the sodium chloride solution was constantly renewed as to be able to analyse the washed-off quantity at given time intervals utilizing HPLC-MS (high performance liquid chromatography-mass spectrometry).

The unmodified expansible hollow parts released over 80% of their total drug load in under 20 seconds; after 1 hour no further release could be detected, i.e. 100% of the total drug load was released. In contrast the expansible hollow parts featuring crescent shaped micro-cavities lost less than 50% of their total drug load in under 20 seconds; after 5 hours there was still detectable drug release from the expansible hollow parts featuring crescent shaped micro-cavities.

1. An expansible hollow part, having at least one opening, which consists of an elastic biocompatible material and which comprises at least one biologically active substance and, optionally, at least one matrix compound, wherein the expansible hollow part is porous and/or comprises micro-cavities in its surface.

2. The expansible hollow part of claim 1, wherein the hollow part is expansible to at least 110% of the circumference of its non-expanded state.

3. The expansible hollow part of claim 1, wherein the hollow part has a wall thickness of smaller than 1 mm in its non-expanded state.

4. The expansible hollow part of claim 1, wherein the surface of the hollow part is substantially impermeable to liquid and/or gas.

5. The expansible hollow part of claim 1, wherein the micro-cavities are elongated.

6. The expansible hollow part of claim 5, wherein the elongated micro-cavities are selected from the group consisting of crescent-shaped furrows, sinuous furrows, circular furrows, elliptical furrows, furrows comprising one or more bends, straight furrows, bifurcated furrows and combinations thereof.

7. The expansible hollow part of claim 5, wherein the elongated micro-cavities have a length of not more than 2 mm.

8. The expansible hollow part of claim 1, wherein the depth of the micro-cavities is between 5 μm and 500 μm.

9. The expansible hollow part of claim 1, wherein the micro-cavities are tilted with respect to the surface of the hollow part.

10. The expansible hollow part of claim 1, wherein the micro-cavities are tilted in direction of the longitudinal axis of the expansible hollow part.

11. The expansible hollow part of claim 1, wherein the micro-cavities are substantially closed when the expansible hollow part is in its non-expanded state.

12. The expansible hollow part of claim 1, wherein the micro-cavities open up when the expansible hollow part is expanded.

13. The expansible hollow part of claim 1, wherein the micro-cavities form fringes protruding from the surface of the expansible hollow part when said expansible hollow part is expanded.

14. The expansible hollow part of claim 1, wherein the expansible hollow part has an inner diameter smaller than 1 cm in its non-expanded state.

15. The expansible hollow part of claim 1, wherein more than 50% of the biologically active substance, and optionally of said matrix compound is located in said micro-cavities.

16. The expansible hollow part according to claim 1, wherein the elastic biocompatible material consists, comprises or essentially consists of a material selected from the group consisting of: natural rubber, polyisoprene, a copolymer of isobutylene and isoprene, a halogenated butyl rubber, a polybutadiene, a styrene-butadiene rubber, a copolymer of polybutadiene and acrylonitrile, a hydrogenated nitrile rubber, a chloroprene rubber, a polychloroprene, latex, a neoprene, a baypren, latex, parylene, polyvalerolactone, poly-ε-decalactone, polylactic acid, polyglycol acid, polylactide, polyglycolide, co-polymer of polylactide and polyglycolide, poly-ε-caprolactone, polyhydroxy butyric acid, polyhydroxybutyrate, polyhydroxyvalerate, polyhydroxybutyrate-co-valerate, poly(1,4-dioxan-2,3-dione), poly(1,3-dioxan-2-one), poly-para-dioxanone, polyanhydride, polymaleicacidanhydride, polyhydroxymethacrylate, fibrin, polycyanoacrylate, polycaprolactondimethylacrylate, poly-β-maleic acid, polycaprolactonbutylacrylate, multiblockpolymers made of oligocaprolactondiole and oligodioxanondiole, polyetherestermultiblockpolymers made from PEG and polybutylenterephtalate, polypivotolactone, poly-glycolic acid trimethylcarbonate polycaprolactonglycolide, poly(g-ethylglutamate), poly(dth-iminocarbonate), poly(dte-co-dt-carbonat), poly(bisphenol A-iminocarbonate), polyorthoester, poly-glycolic acid-trimethylcarbonate, polytrimethylcarbonate polyiminocarbonate, poly(n-vinyl)-pyrrolidone, polyvinylalcohols, polyesteramide, glycolized polyester, polyphosphoester, polyphosphazene, poly(p-carboxyphenoxy)propane], polyhydroxypentanoic acid, polyethylenoxidpropylenoxid, polyurethane, polyurethane comprising amino acids, polyetherester like polyethyleneoxide, polyalkeneoxalate, polyorthoester, lipids, carrageenane, fibrinogen, starch, collagene, protein-based polymers, polyaminoacids, zein, polyhydroxyalkanoate, pectic acid, actinic acid, carboxymethylsulfate, albumine, hyaluronic acid, chitosane, heparanesulfate, heparine, chondroitinsulfate, dextrane, β-cyclodextrine, copolymers comprising PEG and polypropyleneglycole, gummi arabicum, guar, gelatine, collagen-n-hydroxysuccinimide, phospholipids, polyacrylic acid, polyacrylate, polymethylmethacrylate, polybutylmethacrylate, polyacrylamide, polyacrylonitrile, polyamide, polyetheramide, polyethyleneamine, polyimide, polycarbonate, polycarbourethane, polyvinylketone, polyvinylhalogenide, polyvinylidenhalogenide, polyvinylether, polyisobutylene, aromatic compounds comprising a polyvinyl functional group, polyvinylester, polyvinylpyrollidone, polyoxymethylene, polytetramethyleneoxide, polyethylen, polypropylen, polytetrafluorethylen, polyetherurethane, silicon-polyetherurethane, silicon-polyurethane, silicon-polycarbonat-urethane, polyolefin-elastomers, epdm-rubber, fluorosilicone, carboxymethylchitosane, polyaryletheretherketone, polyetheretherketone, polyethylenterephtalate, polyvalerate, carboxymethylcellulose, cellulose, rayon, rayontriacetate, cellulosenitrate, celluloseacetate, hydroxyethylcellulose, cellulosebutyrate, celluloseacetatebutyrate, ethylvinylacetate, polysulfone, epoxy-resin, abs-resin, silicone like polysiloxane, polydimethylsiloxane, polyvinylhalogens, cellulose-ether, cellulose-triacetate, copolymers mixtures and derivatives thereof.

17. The expansible hollow part according to claim 1, wherein the biologically active substance is selected from the group consisting of: a vasoconstrictor, a vasodilatator, a muscle relaxant, an antimycotic, a cytotoxic agent, a prodrug of a cytotoxic agent, a virustatic, a physiological or pharmacological inhibitor of mitogens, a cytostatic, a chemotherapeutic, an adrenocorticostatic, a β-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an angiogenesis inhibitor, an anticholinergic, an enzyme, a coenzyme, an antihistaminic, an antihypertensive, an antihypotensive, an anticoagulant, an antiseptic, an antihemorrhagic, a beta-receptor antagonist, a calcium channel antagonist, an antimyasthenic, an antiphlogistic, an antipyretic, a glucocorticoid, a haemostatic, an immunoglobuline or its fragment, a chemokine, a mitogen, a cell differentiation factor, a hormone, an immunosuppressant, an immunostimulant, a mineralcorticoid, a narcotic, a vector, a peptide, a (para)-sympathicomimetic, a (para)-sympatholytic, a protein, a cell, a sedating agent, a spasmolytic, a wound-healing substance, and combinations thereof.

18. The expansible hollow part according to claim 1, wherein the expansible hollow part comprises at least 0.5 μg of the biologically active substance per square millimetre surface in its non-expanded state.

19. The expansible hollow part according to claim 1, wherein the matrix compound is selected from a group consisting of polyvalerolactone, poly-ε-decalactone, polylactic acid, polyglycolic acid, polylactide, polyglycolide, copolymers of polylactide and polyglycolide, poly-ε-caprolactone, polyhydroxylbutyric acid, polyhydroxybutyrate, polyhydroxyvalerate, polyhydroxybutyrate-co-valerate, poly(1,4-dioxane-2,3-dione), poly(1,3-dioxane-2-one), poly-para-dioxanone, polyanhydride, polymaleicacidanhydride, polyhydroxymethacrylate, fibrin, polycyanoacrylate, polycaprolactondimethylacrylate, poly-b-maleic acid polycaprolactonbutylacrylate, multi-block polymers of oligocaprolactondiole and oligodioxanonediole, polyetherestermultiblockpolymers made of peg und polybutylene terephthalate, polypivotolactone, polyglycolic acid trimethylcarbonate polycaprolactonglycolide, poly(g-ethylglutamate), poly(dth-lminocarbonate), poly(dte-co-dt-carbonate), poly(bisphenol a-iminocarbonate), polyorthoester, polyglycolic acid trimethylcarbonate, polytrimethylcarbonate, polyiminocarbonate, poly(n-vinyl)-pyrrolidone, polyvinylalcohol, polyesteramide, glycolated polyester, polyphosphoester, polyphosphazene, poly[p-carboxyphenoxy)propane], polyhydroxypentanoic acid, polyethyleneoxidepropyleneoxide, polyurethane, polyurethane having amino acids in the backbone, polyetherester like polyethyleneoxide, polyalkeneoxalate, lipid, carrageenane, fibrinogen, starch, collagene, polymers comprising protein, protein, polyaminoacid, synthetic polyaminoacid, zein, polyhydroxyalkaneoate, pectic acid, actinic acid, carboxymethylsulfate, albumin, hyaluronic acid, chitosan und derivatives thereof, heparanesulfate und ist derivatives, heparine, chondroitinsulfate, dextrane, β-cyclodextrine, copolymers of peg and polypropyleneglycol, gummi arabicum, guar, gelatine, collagen-n-hydroxysuccinimide, phospholipid, polyacrylic acid, polyacrylate, polymethyl methacrylate, polybutyl methacrylate, polyacrylamide, polyacrylonitrile, polyamide, polyetheramide, polyethyleneamine, polyimide, polycarbonate, polycarbourethane, polyvinylketone, polyvinylhalogenide, polyvinylidenhalogenide, polyvinylether, polyisobutylene, polyvinyl compounds, polyvinyl ester, polyvinylpyrollidone, polyoxymethylene, polytetramethylene oxid, polyethylene, polypropylene, polytetrafluoroethylene, polyetherurethane, silicone-polyetherurethane, silicone-polyurethane, silicone-polycarbonate-urethane, polyolefin, epdm-rubber, fluorosilicone, carboxymethylchitosane, polyaryletheretherketone, polyetheretherketone, polyethylene terephthalat, polyvalerate, carboxymethylcellulose, rayon, rayontriacetate, cellulose nitrate, cellulose acetate, hydroxyethylcellulose, cellulosebutyrate, celluloseacetatbutyrate, ethylvinylacetate, polysulfone, parylene, epoxy resin, abs-resin, silicone like polysiloxane, polydimethylsiloxane, polyvinylhalogene, cellulose ether, cellulose triacetate, chiosane, N,N-diethylnicotinamide, N-picolylnicotinamide, N-allylnicotinamide, sodium salicylate, 2-methacryloyloxyethyl phosphorylcholine, resorcinol, N,N-dimethylnicotinamide, N-methylnicotinamide, butylurea, pyrogallol, N-picolylacetamide, procaine HCl, nicotinamide, pyridine, 3-picolylamine, sodium ibuprofen, sodium xylenesulfonate, ethyl carbamate, 6-hydroxy-N,N-diethylnicotinamide, sodium p-toluenesulfonate, pyridoxal hydrochloride, 1-methyl-2-pyrrolidone, sodium benzoate, 2-Pyrrolidone, ethylurea, N,N-dimethylacetamide, N-methylacetamide, isoniazid, iopromide, a contrast dye, iobitridol, iohexyl, iomeprol, iopamidol, iopentol, iopromide, ioversol, ioxilan, iotrolan, iodixanol, ioxaglate, and combinations, derivatives and copolymers of combinations thereof.

20. Method of producing an expansible hollow part having at least one opening, which consists of an elastic biocompatible material and which comprises at least one biologically active substance and, optionally, at least one matrix compound, comprising the steps:
(a) expanding the expansible hollow part to at least 110% of its non expanded circumference, and,
(b) contacting the outer surface of the expansible hollow part with at least one biologically active substance and/or at least one matrix compound.

21. The method according to claim 20 further comprising at least one additional step selected from the steps consisting of:
(c) relaxing the expansible hollow part so that its circumference is smaller than the circumference of the expansible hollow part in step (a);
(d) cutting elongated micro-cavities into the elastic biocompatible material; and
(e) mechanically everting the expansible hollow part.

22. The method according to claim 21, wherein the steps (a) and (b) or (b) and (c) are carried out simultaneously.

23. The method according to claim 21, wherein
(i) step (e) is carried out before step (a) and/or after step (b) and/or after step (c);
(ii) steps (a) and (b) are carried out in that order;
(iii) steps (a), (d) and (b) are carried out in that order;
(iv) steps (e), (a) and (b), are carried out in that order; or
(v) steps (b), (e), (b) are carried out in that order, whereby in the first step (b) a different biologically active substance is used than the second step (b).

24. The method according to claim 20, wherein the expansion in step (a) is carried out using an expansible medical device or a mechanical tool.

25. The method according to claim 20, wherein the expansible hollow part is a tubular structure, preferably having two open ends, and comprising a plurality of elongated micro-cavities that are selected from the group consisting of crescent-shaped furrows, sinuous furrows, circular furrows, elliptical furrows, furrows comprising one or more bends, straight furrows, bifurcated furrows and combinations thereof.

26. An expansible hollow part producible by the method of claim 20.

27. A medical device covered at least partially by the expansible hollow part according to claim 1.

28. Medical device according to claim 27, wherein the medical device is selected form the group consisting of a stent, a balloon catheter, a probe, a prosthesis, an endoscope, a pace maker, a heart defibrillator and a perfusion catheter.

29. Medical device according to claim 28, wherein the medical device is a balloon catheter; and wherein the expansible hollow part is covered at least partially by a stent.

30. Medical device according to claim 28, wherein the medical device is a stent; and wherein the stent is on a balloon catheter.

31. Medical device according to claim 28, wherein the balloon catheter comprises a hot balloon, a cold balloon, an occlusion balloon, a valvuloplasty-balloon and/or a protection device.

32. Medical device according to claim 28, wherein the balloon catheter is a perfusion catheter which comprises a hot balloon or a cold balloon.

33. Medical device according to claim 28, wherein the balloon catheter and the expansible hollow part are connected to each other by at least one clamping piece.

34. Medical device according to claim 27, wherein the circumference of the medical device in its non-expanded state is greater than the inner circumference of the expansible hollow part in its non-expanded state.

35. A kit-of-parts comprising at least one expansible hollow part according to claim 1 and at least one medical device.

36. Kit-of-parts according to claim 35, wherein the medical device is selected from the group consisting of a stent, a balloon catheter, a probe, a prosthesis, an endoscope, a pace maker, a heart defibrillator and a perfusion catheter.

37. Kit-of-parts according to claim 35, wherein the circumference of the medical device in its non-expanded state is greater than the inner circumference of the expansible hollow part in its non-expanded state.

38. A method for treatment of a disease or a medical insufficiency comprising treating a subject with a disease or a medical insufficiency selected from the group consisting of a stenosis, a restenosis, a stricture, a defective bypass craft, a thrombosis, a dissection, a tumor, a calcification, an arteriosclerosis, an inflammation, an autoimmune response, a necrosis, an injured anastomosis, a lesion, an allergy, a wart, a hyperproliferation, an infection, a scald, an edema, a coagulation, a cicatrization, a burn, a frostbite and a lymphangitis, with a therapeutic device comprising the expansible hollow part according to claim 1.

39. The method of claim 38, wherein the therapeutic device comprises a balloon catheter.

40. (canceled)
